(19) United States
(12) Patent Application Publication
(10) Pub. No.: US 2024/0131231 A1
(43) Pub. Date: Apr. 25, 2024

(54) NITRIC OXIDE-RELEASING COMPOSITIONS AND APPLICATIONS THEREOF

(71) Applicant: University of Georgia Research Foundation, Inc., Athens, GA (US)

(72) Inventors: Hitesh HANDA, ATHENS, GA (US); Sean P. HOPKINS, ATHENS, GA (US)

(21) Appl. No.: 18/264,094

(22) PCT Filed: Feb. 1, 2022
(86) PCT No.: PCT/US2022/014689

§ 371 Date: Aug. 3, 2023

Related U.S. Application Data
(60) Provisional application No. 63/145,760, filed on Feb. 4, 2021.

(51) Int. Cl. A61L 29/16; A01N 25/04; A01N 59/00; A01P 1/00; A61L 29/06; A61L 29/08

(52) U.S. Cl. CPC A61L 29/16; A01N 25/04; A01N 59/00; A01P 1/00; A61L 29/06; A61L 29/085

(57) ABSTRACT

Described herein are compositions comprising a nitric oxide releasing material comprising a (i) a polysiloxane network and (ii) a plurality of nitric oxide-donating moieties covalently bonded to the polysiloxane network; and (b) a silicone oil. Also described herein are articles composed of the compositions described herein and methods for using the same. The compositions described herein provide highly sustained, long-term nitric oxide release that have numerous biological activities such as, for example, preventing localized platelet activation, fibrinogen adhesion, and biofilm formation for a prolonged period of time.

### CROSS-REFERENCE TO RELATED APPLICATIONS

This application claims the benefit of and priority to co-pending U.S. Provisional Patent Application No. 63/145,760, filed on Feb. 4, 2021, the contents of which are incorporated by reference herein in their entireties.

### STATEMENT REGARDING FEDERALLY SPONSORED RESEARCH OR DEVELOPMENT

This invention was made with government support under award R01HL134899 awarded by the National Institutes of Health and award 75D3011P06553 awarded by Centers for Disease Control and Prevention. The government has certain rights in the invention.

### BACKGROUND

Long-term, indwelling medical devices such as vascular catheters, chest ports, stents, and pacemakers are vital for the diagnosis, mitigation, and treatment of a substantial number of diseases and ailments. However, currently available devices frequently fail due to catastrophic events commonly associated with medical device use including infection, biofouling, and device-induced thrombosis. Medical device-related infections represent a substantial number of nosocomial infections—in critically ill patients, vascular catheter use accounts for 87% of bloodstream infections, urinary catheter use accounts for 95% of urinary tract infections, and mechanical ventilation accounts for 86% of pneumonia infections.¹ Vascular access used for the administration of intravenous therapies, antibiotic treatments, and blood transfusions constitutes a breach in the barrier between the outside environment and the bloodstream, further increasing the risk of local and systemic infections including catheter-related bloodstream infections (CRBSOs), septic thrombophlebitis, endocarditis, and other metastatic infections.² The current standard for controlling infection is antibiotic treatment, but due to the emergence of antibiotic resistance coupled with the persistent presence of microbial biofilms, which readily form on foreign surfaces and exhibit defensive mechanisms including poor antibiotic penetration, limited nutrient uptake, and adaptive stress responses,³ alternative means to prevent and combat infection are needed.

Beyond the issues of infection, when medical devices are exposed to blood, proteins rapidly adsorb and a complex sequence of biochemical reactions is triggered, ultimately resulting in thrombus formation. Clots formed on the surface can totally occlude the device, obstruct device function, and can break off and move further downstream, potentially causing pulmonary embolism or myocardial infarction.⁴ Venous thromboembolism is one of the most prevalent complications associated with indwelling vascular access devices, reportedly occurring in 11-25% of critically ill patients with central venous catheters.⁵ Such complications can result in increased medical costs, extended hospitalization, or increased morbidity.⁶ To maintain device patency, clinicians currently administer anticoagulation therapies to prevent thrombosis, but systemic anticoagulation requires a careful balance between over- and under-administration to prevent clotting while avoiding hemorrhaging.⁷ For indwelling vascular access devices, heparin-based lock solutions therapies are regularly used to prevent device occlusion but can lead to complications such as low platelet counts, internal bleeding, and thrombocytopenia.^{8, 9} Systemic anticoagulation also fails to prevent the adsorption of plasma proteins such as fibrinogen, a central player in the formation of dense fibrin networks and an anchor exploited by bacteria to increase adhesion and biofilm development.¹⁰⁻¹²

To reduce the frequency of medical device-associated infections, researchers have begun to develop different antimicrobial surface modifications (ex. antibiotic-releasing surfaces, silver-based coatings) to counteract bacterial colonization.^{13, 14} However, the development of antibiotic resistance remains a significant limiting factor of antibiotic-eluting surfaces.¹⁴ Moreover, commercial catheters impregnated with antibiotics and antiseptic agents (ex. silver-coated) have shown mixed clinical efficacy against infection.¹⁵⁻¹⁹ In fact, silver nanoparticle-impregnated central venous catheters demonstrated no change in the rate of acquired CRBSIs when compared to controls.²⁰ In addition, silver-eluting central venous catheters invoke a pro-thrombotic response as a result of rapid thrombin generation, restricting its applications for blood-contacting medical devices.²¹

To prevent surface-induced thrombosis, hemocompatible surface modifications aim to disrupt the coagulation cascade by preventing protein adsorption, impeding platelet adhesion and activation, or inhibiting thrombin-mediated reactions. Antithrombotic surface modifications can be broken down into two categories based on the method of increasing hemocompatibility: (1) passive surface strategies, which minimize contact with blood components, and (2) active surface strategies, which store and release antithrombotic or fibrinolytic agents that directly interrupt coagulation.²² However, due to the complexity of the physiological components and mechanisms that lead to medical device-induced thrombosis, a long-term antithrombotic solution for medical devices will likely require a combination of strategies.²³ Routine failure of blood-contacting devices can be attributed to the material's inability to replicate the multifunctional antithrombotic functionality of the surrounding vasculature.²⁴ Despite tremendous efforts that have been made, a single platform that exhibits comprehensive hemocompatibility has yet to be fabricated.²⁵

### SUMMARY

Described herein are compositions comprising a nitric oxide releasing material comprising a (i) a polysiloxane network and (ii) a plurality of nitric oxide-donating moieties covalently bonded to the polysiloxane network; and (b) a silicone oil. Also described herein are articles composed of the compositions described herein and methods for using the same. The compositions described herein provide highly sustained, long-term nitric oxide release that have numerous biological activities such as, for example, preventing localized platelet activation, fibrinogen adhesion, and biofilm formation for a prolonged period of time.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows how catheter-related infections can occur within intravascular catheters. Biofilms have a chance to form at the device insertion interface, causing infections that cannot be treated with traditional antibiotic therapies. Thrombus formation can also trap bacteria, which have the potential to form clots that detach and migrate to other parts of the body.

FIG. 2 shows hemostatic pathways for thrombus formation.

FIG. 3 shows the CDC bioreactor setup used to evaluate the antimicrobial activity of the materials over 28 days.

FIG. 4 shows the fabrication of SNAP-immobilized, silicone oil-infused PDMS (LI-NO-PDMS). The NO donor SNAP was covalently bound to hydroxy-terminated PDMS prior to infusion of silicone oil (50 cst).

FIGS. 5A-5B show liquid infusion optimization of NO-PDMS materials. (A) Swelling ratio of NO-PDMS materials within silicone oil. The optimized swelling time was found to be 8 h, which gave a swelling ratio of 2.0. (B) Sliding angle of NO-PDMS and LI-NO-PDMS surfaces when being kept in PBS at 37° C. over 7 days. LI-NO-PDMS shows significantly lower sliding angle measurements compared to NO-PDMS during the entire incubation period (p<0.001).

FIGS. 6A-6B show (A) NO release characteristics of LI-NO-PDMS and NO-PDMS materials when placed within PBS containing 0.01M EDTA at 37° C. over the course of 30 days. (B) SNAP leaching (mg of SNAP per mg of polymer) from both NO-PDMS and LI-NO-PDMS polymers in PBS at 37° C. for 48 h.

FIGS. 7A-7D show the antimicrobial results of LI-NO-PDMS surfaces against MRSA after 24 h (A), 7 days (B), 14 days (C), and 28 days (D) using a 24 h adhesion assay and CDC biofilm reactors (for studies >24 h). Significance in bacterial reduction between groups were indicated according to ANOVA statistical analysis (*=p<0.05, **=p<0.01, ***=p<0.001).

FIGS. 8A-8D show the antimicrobial results of LI-NO-PDMS surfaces against P. aeruginosa after 24 h (A), 7 days (B), 14 days (C), and 28 days (D) using a 24 h adhesion assay and CDC biofilm reactors (for studies >24 h). Significance in bacterial reduction between groups were indicated according to ANOVA statistical analysis (*=p<0.05, **=p<0.01, ***=p<0.001).

FIGS. 9A-9N show (A) the quantification of adsorbed fibrinogen to PDMS and LI-NO-PDMS polymer surfaces and (B) platelet adhesion measurements after 2 h of in vitro porcine platelet rich plasma exposure. Images of fluorescently labeled fibrinogen (C-F) show that while NO-PDMS samples show an increased level of protein adsorption, LI-PDMS and LI-NO-PDMS surfaces showed reduced levels of protein adsorption. (G-N) SEM images were also taken after porcine whole blood exposure for 60 s, showing a noticeable reduction in activated platelets and fibrin formation on LI-NO-PDMS surfaces compared to control PDMS surfaces. Red arrows indicate activated platelets, and green arrows indicate inactivated platelets.

FIG. 10 shows the relative cell viability of BJ fibroblasts and HUVECs treated with leachates gathered from prepared films following 24 h in physiological conditions. Data represented as mean ±SD.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### DEFINITIONS

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” “having”, “has,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a polysiloxane” includes, but is not limited to, mixtures or combinations of two or more such polysiloxanes, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. The term “alkyl” also refers to alklylene groups represented by the general formula —(CHR)ₙ—, where R is an alky group as defined above (e.g., methyl, ethyl, etc.) and n is an integer from 1 to 20. Examples of alklylene groups include, but are not limited to, methylene, ethylene, propylene, and the like.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, having from one to ten carbons, or from one to six carbon atoms in its backbone structure. Likewise, “lower alkenyl” and “lower alkynyl” have similar chain lengths. In embodiments described in the present application, preferred alkyl groups are lower alkyls. In some embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In some embodiments, the “alkylthio” moiety is represented by one of -S-alkyl, -S-alkenyl, and -S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl”, refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxyl, such as can be represented by one of -O-alkyl, -O-alkenyl, and -O-alkynyl. Aroxy can be represented by -O-aryl or O-heteroaryl, wherein aryl and heteroaryl are as defined below. The alkoxy and aroxy groups can be substituted as described above for alkyl.

The terms “amine” and “amino” are art-recognized and refer to both unsubstituted and substituted amines, e.g., a moiety that can be represented by the general formula:

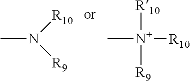

wherein R₉, R₁₀, and R′₁₀ each independently represent a hydrogen, an alkyl, an alkenyl, —(CH₂)ₘ-R₈ or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R′₁₀) each independently represent a hydrogen, an alkyl or cycloalkyl, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The term “amido” is art-recognized as an amino-substituted carbonyl and includes a moiety that can be represented by the general formula:

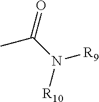

wherein R₉ and R₁₀ are as defined above.

“Aryl”, as used herein, refers to C₅-C₁₀ -membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl”, as used herein, refers to an alkyl group substituted with an aryl group (e.g., an aromatic or heteroaromatic group).

The term “carbocycle”, as used herein, refers to an aromatic or non-aromatic ring in which each atom of the ring is carbon.

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C₁-C₁₀) alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, and —CN.

The term “carbonyl” is art-recognized and includes such moieties as can be represented by the general formula:

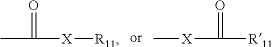

wherein X is a bond or represents an oxygen or a sulfur, and R₁₁ represents a hydrogen, an alkyl, a cycloalkyl, an alkenyl, an cycloalkenyl, or an alkynyl, R′₁₁ represents a hydrogen, an alkyl, a cycloalkyl, an alkenyl, an cycloalkenyl, or an alkynyl. Where X is an oxygen and R₁₁ or R′₁₁ is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “monoester” as used herein refers to an analogue of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “heteroatom” as used herein means an atom of any element other than carbon or hydrogen. Examples of heteroatoms include, but are not limited to boron, nitrogen, oxygen, phosphorus, sulfur and selenium. Other heteroatoms include silicon and arsenic.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms (for example, 1-14 carbon atoms), and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various aspects, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

The term “copolymer” as used herein, generally refers to a single polymeric material that is comprised of two or more different monomers. The copolymer can be of any form, such as random, block, graft, etc. The copolymers can have any end-group, including capped or acid end groups.

The term “prevent” or “preventing” as used herein is defined as eliminating or reducing the likelihood of the occurrence of one or more symptoms of a disease or disorder (e.g., biofilm formation) when using the compositions as described herein when compared to a control where the composition is not used.

### Nitric Oxide Releasing Materials and Articles and Methods of Making and Uses Thereof

Described herein are nitric oxide-releasing compositions. In one aspect, the compositions comprise (a) a nitric oxide releasing material comprises (i) a polysiloxane network and (ii) a plurality of nitric oxide-donating moieties covalently bonded to the polysiloxane network and (b) a silicone oil. The compositions described herein provide highly sustained, long-term nitric oxide release that have numerous biological activities.

In one aspect, the polysiloxane network in the nitric oxide releasing material is the reaction product between polysiloxane and an amine-functionalized crosslinker. In one aspect, the polysiloxane comprises one or more functional groups that can react with the amine-functionalized crosslinker. In one aspect, the polysiloxane includes two or more hydroxyl and/or amino groups. In another aspect, the polysiloxane is terminated with a hydroxyl group, which is referred to herein as a “hydroxy-terminated polysiloxane.” For example, if the polysiloxane is linear, then each end of the polysiloxane is terminated with a hydroxyl group. In other aspects, when the polysiloxane is branched, then each branch of the polysiloxane is terminated with a hydroxyl group.

In one aspect, the polysiloxane is a polydimethylsiloxane, a polydiethylsiloxane, a polydipropylsiloxane, or a polydiphenylsiloxane. In another aspect, the polysiloxane used to make the polysiloxane network is a polydimethylsiloxane, a polydiethylsiloxane, a polydipropylsiloxane, or a polydiphenylsiloxane terminated with a hydroxyl group. In one aspect, the polysiloxane has a kinematic viscosity of about 2,500 cSt to about 4000 cSt, or about 2,500 cSt, 2,550 cSt, 2,600 cSt, 2,650 cSt, 2,700 cSt, 2,750 cSt, 2,800 cSt, 2,850 cSt, 2,900 cSt, 2,950 cSt, 3,050 cSt, 3,100 cSt, 3,150 cSt, 3,200 cSt, 3,250 cSt, 3,300 cSt, 3,350 cSt, 3,400 cSt, 3,450 cSt, 3,500 cSt, 3,550 cSt, 3,600 cSt, 3,650 cSt, 3,700 cSt, 3,750 cSt, 3,800 cSt, 3,850 cSt, 3,900 cSt, 3,950 cSt, or 4,000 cSt, where any value can be a lower and upper endpoint of range (e.g., 2,550 cSt to 3,600 cSt).

In one aspect, the amine-functionalized crosslinker includes one or more groups that react with the polysiloxane to form the polysiloxane network. In one aspect, amine-functionalized crosslinker is an amino silane compound. In one aspect, when the polysiloxane is terminated with hydroxyl groups, the hydroxyl group reacts with the silane group of the amino silane compound. In one aspect, the amino silane compound has the structure:

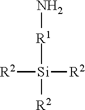

where R¹ is selected from a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy; where each occurrence of R² is hydroxy or alkoxy. In one aspect, R¹ is a C₁-C₁₀ alkyl group such as, for example, methylene, ethylene, propylene, butylene, and the like.

A plurality of nitric oxide-donating moieties is covalently bonded to the polysiloxane network. Not wishing to be bound by theory, the polysiloxane network includes a plurality of amino groups derived from the amine-functionalized crosslinker. The amino groups can further react with additional compounds that covalently bond nitric oxide-donating moieties or precursors thereof to produce the polysiloxane network. In one aspect, a compound possessing one or more nitric oxide groups can be reacted directly with the polysiloxane network to produce the nitric oxide releasing material.

In other aspects, the polysiloxane network can be reacted with a compound that possesses one or more groups that are a precursor to the nitric oxide releasing material. In one aspect, the compound possesses one or more sulfur groups that can be subsequently nitrosylated. In one aspect, the polysiloxane network is reacted with a thiolactone. Not wishing to be bound by theory, the amino groups present in the polysiloxane network react with the thiolactone, where the thiolactone ring-opens to produce a free thiol group or ion. In one aspect. In one aspect, the thiolactone has the structure:

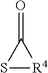

where R⁴ is a substituted or unsubstituted C₁-C₁₂ alkyl (e.g., methylene, ethylene, propylene, butylene).

In another aspect, the thiolactone has the structure:

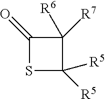

where each occurrence of R⁵ is independently hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, or a substituted or unsubstituted C₁-C₆ heteroalkoxy group;

R⁶ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, or a substituted or unsubstituted C₁-C₆ heteroalkoxy group; and

R⁷ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, a substituted or unsubstituted C₁-C₆ heteroalkoxy group, or an amide group of the formula —NHC(O)R⁸, wherein R⁸ is a substituted or unsubstituted C₁-C₆ alkyl group, substituted or unsubstituted C₁-C₆ heteroalkyl group.

In one aspect, the thiolactone is N-acetylcysteine thiolactone, N-acetyl- homocysteine thiolactone, homocysteine thiolactone, butyryl-homocysteine thiolactone, or any combination thereof.

In one aspect, the nitric oxide releasing material includes a plurality of —S—NO groups. For example, when the polysiloxane network is reacted with a thiolactone as provided above, a plurality of thiol groups is produced. The thiol groups can subsequently be nitrosylated by reacting the free thiol groups with a nitrosylating agent. In one aspect, the nitrosylating agent is t-butyl nitrite, isopentyl nitrite, isobutyl nitrite, amyl nitrite, or cyclohexyl nitrite

In certain aspects, nitrosylation of the thiol group can be performed in the presence of an acid catalyst. In one aspect, the organic acid is an organic sulfonic acid having the formula RS(O)₂OH, where R is an alkyl group or aryl group as defined herein. In one aspect, R is an aryl group substituted with a C₁-C₂₀ alkyl group. In one aspect, the sulfonic acid includes, but is not limited to, dodecylbenzene sulfonic acid, dinonylnaphthalenedisulfonic acid, or 4-octylbenzenesulfonic acid. In other aspects, the organic acid can be acetic acid, formic acid, or lactic acid. In one aspect, the organic acid used is from about 0.1 weight percent to about 2 weight percent of the polysiloxane network, or about 0.1 weight percent, 0.1 weight percent, 0.2 weight percent, 0.3 weight percent, 0.4 weight percent, 0.5 weight percent, 0.6 weight percent, 0.7 weight percent, 0.8 weight percent, 0.9 weight percent, 1.0 weight percent, 1.1 weight percent, 1.2 weight percent, 1.3 weight percent, 1.4 weight percent, 1.5 weight percent, 1.6 weight percent, 1.7 weight percent, 1.8 weight percent, 1.9 weight percent, or 2.0 weight percent, where any value can be a lower and upper endpoint of range (e.g., 0.8 weight percent to 1.2 weight percent).

The nitric oxide-donating moieties covalently bonded to the polysiloxane network can vary depending upon the selection of starting materials used to produce the nitric oxide releasing material. In one aspect, the nitric oxide-donating moiety is a S-nitrosothiol. In another aspect, the S-nitrosothiol is a residue of S-nitroso-N-acetyl-penicillamine, S-nitroso-N-acetyl cysteine, S-nitroso-N-acetyl cysteamine, S-nitrosoglutathione, methyl S-nitrosothioglycolate, and a derivative thereof. In another aspect, the nitric oxide-donating moiety is a diazeniumdiolate. In one aspect, the diazeniumdiolate is diazeniumdiolated dibutylhexanediamine or a derivative thereof.

In one aspect, the nitric oxide-donating moiety has a structure according to the following structure:

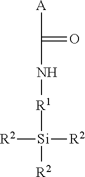

where A is a nitric oxide donor; where R₁ is selected from a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy; where each occurrence of R² is independently a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, a substituted or unsubstituted C₁-C₂₀ heteroalkoxy, or a bond to a polysiloxane in the plurality of polysiloxanes wherein at least two occurrences of R² are a bond to the polysiloxane network via a siloxane bond (e.g., —O—Si—O—). In one aspect, A is a S-nitrosothiol group. In another aspect, A has the structure below

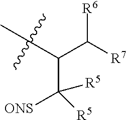

where R⁵, R⁶, and R⁷ are as defined above.

In one aspect, the nitric oxide releasing material can have the structure below:

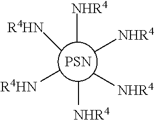

where PSN is the polysiloxane network and R⁴ is a nitric oxide-donating moiety. In one aspect, R⁴ is a S-nitrosothiol group. In another aspect, R⁴ is a residue S-nitroso-N-acetyl-penicillamine, S-nitroso-N-acetyl cysteine, S-nitroso-N-acetyl cysteamine, S-nitrosoglutathione, methyl S-nitrosothioglycolate, or a derivative thereof. In this aspect, the plurality of amino groups derived from the amine-functionalized crosslinker are covalently bonded to the nitric oxide-donating moiety.

The amount of the nitric oxide-donating moieties present in the nitric oxide releasing material can vary. In one aspect, the nitric oxide-donating moieties are present in an amount from about 0.15 micromoles per milligram of the polysiloxane network to about 0.80 micromoles per milligram of the polysiloxane network, or about 0.15 micromoles per milligram, 0.20 micromoles per milligram, 0.25 micromoles per milligram, 0.30 micromoles per milligram, 0.35 micromoles per milligram, 0.40 micromoles per milligram, 0.45 micromoles per milligram, 0.50 micromoles per milligram, 0.55 micromoles per milligram, 0.60 micromoles per milligram, 0.65 micromoles per milligram, 0.70 micromoles per milligram, 0.75 micromoles per milligram, or 0.80 micromoles per milligram, where any value can be a lower and upper endpoint of range (e.g., 0.30 micromoles per milligram to 0.70 micromoles per milligram).

The compositions described herein include a silicone oil. Not wishing to be bound by theory, varying the amount of the silicone oil can modulate the release of nitric oxide from the nitric oxide releasing material. In one aspect, the nitric oxide releasing material and silicone oil are in an amount sufficient such that the composition has a swelling ratio of from about 0.5 to about 3, where the swelling ratio is defined by the mass of a measured portion of silicone swelled in oil divided by its original mass before swelling. In another aspect, the swelling ratio is 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0, where any value can be a lower and upper endpoint of range (e.g., 1.8 to 2.2). The viscosity of the silicone oil can also vary depending upon the application of the composition. In one aspect, the silicone oil has a viscosity of from about 10 cSt to about 500 cSt, or about 10 cSt, 25 cSt, 50 cSt, 75 cSt, 100 cSt, 125 cSt, 150 cSt, 175 cSt, 200 cSt, 225 cSt, 250 cSt, 275 cSt, 300 cSt, 350 cSt, 375 cSt, 400 cSt, 425 cSt, 450 cSt, 475 cSt, or 500 cSt, where any value can be a lower and upper endpoint of range (e.g., 25 cSt to 100 cSt).

The compositions described herein are slippery materials, which makes them useful and effective in indewelling medical devices such as, for example, catheters. The compositions described herein possess sliding angles under physiological conditions for extended periods of time. In one aspect, the sliding angles of the compositions described herein stored at 37° C. in PBS were from 15 degrees to 30 degrees over a seven day period.

The compositions described herein can be formulated in different ways depending upon the application of the composition. In one aspect, the composition is an admixture of the nitric oxide releasing material and silicone oil. In this aspect, the nitric oxide releasing material and silicone oil are intimately mixed such that the nitric oxide releasing material is evenly (i.e., homogeneously) dispersed throughout the silicone oil. In other aspects, the nitric oxide releasing material is impregnated with the silicone oil. In one aspect, a coating of silicone oil is applied to a coating of the nitric oxide releasing material, where the silicone oil permeates (i.e., impregnates) into the nitric oxide releasing material.

By varying the relative amount of the nitric oxide releasing material and silicone oil, rate of release of the nitric oxide releasing material from the composition can be modified. In certain applications, it is desirable to have sustained release of nitric oxide from the composition under physiological conditions. In one aspect, nitric oxide is released from the composition for at least 30 days, at least 45 days, at least 60 days, at least 75 days, or at least 90 days at 37° C. In another aspect, the amount of nitric oxide released from the composition is at least 0.5×10¹⁰ mol cm⁻² min⁻¹ over a period of 5 days, 10 days, 15 days, 20 days, 25 days, or 30 days. In another aspect, the amount of nitric oxide released from the composition is from about 0.5×10 10 mol cm⁻² min⁻¹ to about 2.0×10¹⁰ mol cm⁻² min⁻¹ over a period of 5 days, 10 days, 15 days, 20 days, 25 days, or 30 days.

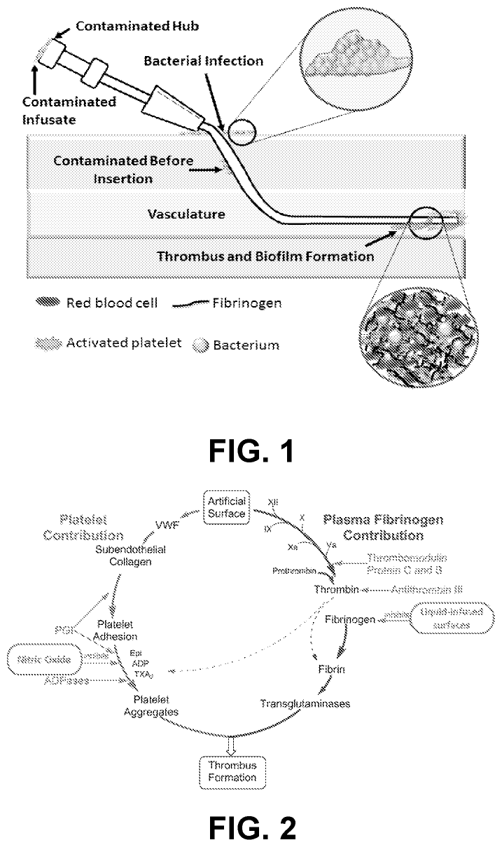

The use of biomedical devices is inevitable in hospital-based care. Unfortunately, it is also one of the leading causes of nosocomial infections. There are multiple factors that contribute to the risk of infection, including the method and duration of catheterization, quality of catheter care, and host susceptibility. The colonization of bacteria can cause infection through a number of pathways (FIG. 1), the two most common being colonization at the insertion site by microorganisms that move through the transcutaneous part of the dermal tunnel surrounding the catheter and colonization in the intraluminal surface. Plasma proteins binding on the surface of intravascular catheters worsen this problem as they can promote bacterial adhesion. Fibrinogen is a positive acute phase protein related to blood infection, inflammatory disease, and tissue damage, ultimately causing patient mortality via trauma coagulopathy.

The compositions described herein are useful in applications where it is desirable to reduce or prevent biofouling (e.g., bacterial adhesion, platelet formation, etc.) of implantable medical devices. Implantable medical devices are a leading cause of infection such as nosocomial infections. Implantable devices coated with or constructed of the compositions described herein can reduce or prevent biofouling in a subject when the device is introduced into the subject. In one aspect, the compositions described herein can reduce or prevent bacterial growth on a surface of an implantable device. In another aspect, the compositions described herein can reduce or prevent biofilm formation on a surface of an implantable device.

In another aspect, the compositions described herein can reduce or prevent fibrinogen formation on a surface of an implantable device. Fibrinogen, a key coagulation protein, rapidly adsorbs to foreign surfaces and activates platelets. Fibrinogen contains multiple binding sites for platelet integrin a α_{IIb}β₃ (GPIIbIIIa). These fibrinogen—α_{IIb}β₃ interactions play a significant role in platelet adhesion, activation, and aggregation that ultimately leads to a clot formation. To prevent thrombosis, a surface that can resist both fibrinogen binding and platelet activation is highly desirable; however, no surface reported to date is able to accomplish this.

Hemocompatibility of blood-contacting biomaterials is highly dependent on the suppression of both the contact coagulation pathway (i.e., fibrin formation) and the activation of circulating platelets (FIG. 2). The nonthrombogenic surfaces that are currently available are designed to inhibit fibrin formation, but these surfaces do not prevent the parallel hemostatic pathway of platelet adhesion/activation and therefore are minimally effective. Thrombus and biofilm formation are highly related, where fouling of these devices is the most common clinical complication, either through protein adsorption leading to thrombus formation or bacterial adhesion resulting in infection. The compositions described herein can prevent platelet adhesion on a surface of an article.

In one aspect, the implantable device is a urinary catheter, artificial heart valve, a vascular catheter, a graft, or a stent. In other aspects, the device is intended to contact human blood or tissue. In one aspect, the device is a hemodialysis device or a component thereof.

The compositions described herein can be incorporated into devices in a number of different ways. In one aspect, the devices can be coated with a composition as described herein. In one aspect, the coating composition can include an admixture of the nitric oxide releasing material and silicone oil. In another aspect, a coating of the nitric oxide releasing material can be applied to a surface of the device to produce a first coating followed by applying a coating of silicone oil on the first coating. The coating of the device can be performed using techniques known in the art such as, for example, spraying or dipping the device with the nitric oxide releasing material and silicone oil. The coating thickness can vary as well depending upon the device and application selected. In one aspect, the nitric oxide releasing material coating has a thickness of from about 0.1 mm to about 5 mm, or about 0.1 mm, 0.5 mm, 1.0 mm, 1.5 mm, 2.0 mm, 2.5 mm, 3.0 mm, 3.5 mm, 4.0 mm, 4.5 mm, or 5.0 mm, where any value can be a lower and upper endpoint of range (e.g., 0.5 mm to 3.0 mm).

In other aspects, the compositions described herein can be used to fabricate a device. For example, when the device is composed of rubber or includes a rubber component, the rubber can be prepared such that the composition described herein is dispersed throughout the rubber to produce a nitric oxide releasing rubber. Once the nitric oxide releasing rubber has been produced, it can be used to produce devices (e.g., medical implantable devices).

### Aspects

Aspect 1. A composition comprising
(a) a nitric oxide releasing material comprising a (i) a polysiloxane network and (ii) a plurality of nitric oxide-donating moieties covalently bonded to the polysiloxane network; and
(b) a silicone oil.

Aspect 2. The composition of Aspect 1, wherein the nitric oxide-donating moiety comprises an S-nitrosothiol

Aspect 3. The composition of Aspect 1, wherein the nitric oxide-donating moiety is a residue of S-nitroso-N-acetyl-penicillamine, S-nitroso-N-acetyl cysteine, S-nitroso-N-acetyl cysteamine, S-nitrosoglutathione, methyl S-nitrosothioglycolate, and a derivative thereof.

Aspect 4. The composition of any of Aspects 1-3, wherein the polysiloxane network comprises a polysiloxane crosslinked with an amine-functionalized crosslinker.

Aspect 5. The composition of Aspect 4, wherein the polysiloxane comprises a polydimethylsiloxane, a polydiethylsiloxane, a polydipropylsiloxane, or a polydiphenylsiloxane.

Aspect 6. The composition of Aspect 4 or 5, wherein the polysiloxane has a kinematic viscosity of about 2,500 cSt to about 4,000 cSt.

Aspect 7. The composition of any of Aspects 1-6, wherein the nitric oxide-releasing material is produced the method comprising:
crosslinking a polysiloxane with an amine-functionalized crosslinker to produce a polysiloxane network
covalently attaching a thiolactone to the polysiloxane network to produce a thiol-functionalized polysiloxane network; and
nitrosating a thiol group in the thiol-functionalized polysiloxane network in the presence of an organic acid to produce the nitric oxide-releasing material.

Aspect 8. The composition of Aspect 7, wherein the organic acid comprises an organic sulfonic acid.

Aspect 9. The composition of Aspect 7, wherein the organic acid comprises dodecylbenzene sulfonic acid, dinonylnaphthalenedisulfonic acid, 4-octylbenzenesulfonic acid, acetic acid, formic acid, or lactic acid.

Aspect 10. The composition of Aspect 7, wherein the organic acid is dodecylbenzene sulfonic acid.

Aspect 11. The composition of Aspect 7, wherein the organic acid is in the amount of from about 0.1 weight percent to about 2 weight percent of the polysiloxane network.

Aspect 12. The composition of Aspect 7, wherein the polysiloxane comprises a hydroxy-terminated polysiloxane.

Aspect 13. The composition of Aspect 7, wherein the amine-functionalized crosslinker has the structure

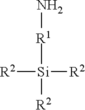

where R¹ is selected from a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy;
where each occurrence of R² is hydroxy or alkoxy.

Aspect 14. The composition of Aspect 13, wherein each occurrence of R² is a hydroxy, methoxy or ethoxy.

Aspect 15. The composition of Aspect 13, wherein R¹ is a substituted or unsubstituted C1-012 alkyl or a substituted or unsubstituted C₁-C₁₂ aminoalkyl.

Aspect 16. The composition of Aspect 13 or 14, wherein R¹ is methylene, ethylene, propylene, or butylene.

Aspect 17. The composition of Aspect 7, wherein the thiolactone has the structure

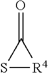

where R⁴ is a substituted or unsubstituted C₁-C₁₂ alkyl.

Aspect 18. The composition of Aspect 7, wherein the thiolactone has the structure

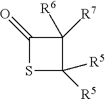

where each occurrence of R⁵ is independently hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, or a substituted or unsubstituted C₁-C₆ heteroalkoxy group;
R⁶ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, or a substituted or unsubstituted C₁-C₆ heteroalkoxy group; and

R⁷ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, a substituted or unsubstituted C₁-06 heteroalkoxy group, or an amide group of the formula —NHC(O)R⁸, wherein R⁸ is a substituted or unsubstituted C₁-C₆ alkyl group, substituted or unsubstituted C1-06 heteroalkyl group.

Aspect 19. The composition of Aspect 7, wherein the thiolactone is selected from the group consisting of N-acetylcysteine thiolactone, N-acetyl-homocysteine thiolactone, homocysteine thiolactone, and butyryl-homocysteine thiolactone.

Aspect 20. The composition of any of Aspects 1-19, wherein the nitric oxide-donating moiety is a residue of S-nitroso-N-acetyl-penicillamine, S-nitroso-N-acetyl cysteine, S-nitroso-N-acetyl cysteamine, S-nitrosoglutathione, or methyl S-nitrosothioglycolate.

Aspect 21. The composition of any of Aspects 1-20, wherein the nitric oxide-donating moieties are present in an amount from about 0.15 micromoles per milligram of the polymer matrix to about 0.80 micromoles per milligram of the polysiloxane network.

Aspect 22. The composition of any of Aspects 1-21, wherein the silicone oil has a viscosity of from about 10 cSt to about 500 cSt.

Aspect 23. The composition of any of Aspects 1-21, wherein nitric oxide releasing material and silicone oil are in an amount sufficient such that the composition has a swelling ratio of from about 0.5 to about 3.

Aspect 24. The composition of any of Aspects 1-23, wherein nitric oxide is released from the composition for at least 30 days at 37 ° C.

Aspect 25. The composition of any of Aspects 1-23, wherein the composition comprises an admixture of the nitric oxide releasing material and silicone oil.

Aspect 26. The composition of any of Aspects 1-23, wherein the nitric oxide releasing material is impregnated with the silicone oil.

Aspect 27. An article comprising at least one surface, wherein the at least one surface is coated with the composition in any one of Aspects 1-26.

Aspect 28. An article comprising one or more components fabricated with the composition in any one of Aspects 1-26.

Aspect 29. The article of Aspects 27 or 28, wherein the article comprises a medical device.

Aspect 30. The article of Aspect 29, wherein the device is an implantable device.

Aspect 31. The article of Aspect 29, wherein the device is selected from the group consisting of: a vascular catheter, a urinary catheter, other catheters, a coronary stent, a wound dressing, and a vascular graft.

Aspect 32. A method of making an article, the method comprising
(a) applying a nitric oxide releasing material to at least one surface of the article to produce a first coated article; and
(b) applying silicone oil to the first coated article.

Aspect 33. The method of Aspect 32, wherein the nitric oxide releasing material has a thickness of from about 0.1 mm to about 5 mm.

Aspect 34. The method of Aspect 32, wherein the first coated article is dipped into the silicone oil from about 1 hour to about 12 hours.

Aspect 35. A method of reducing or preventing bacterial growth on a surface of an article, the method comprising applying the composition in any one of Aspects 1-26 to the surface.

Aspect 36. A method of reducing or preventing biofilm formation on a surface of an article, the method comprising applying the composition in any one of Aspects 1-26 to the surface.

Aspect 37. A method of reducing preventing fibrinogen formation on a surface of an article, the method comprising applying the composition in any one of Aspects 1-26 to the surface.

Aspect 38. A method of reducing or preventing platelet adhesion on a surface of an article, the method comprising applying the composition in any one of Aspects 1-26 to the surface.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Materials and Methods

### Materials

N-Acetyl-D-penicillamine (NAP), hydroxy-terminated PDMS (2550-3570 cSt), toluene, dibutyltin dilaurate, tent-butyl nitrite, pyridine, acetic anhydride, chloroform, anhydrous magnesium sulfate, (3-amino-propyl) trimethoxysilane, 1,4,8,11-tetraazacyclotetradecane (cyclam), hexanes, dodecylbenzene sulfonic acid, ethylenediaminetetraacetic acid (EDTA), and hydrochloric acid were purchased from Sigma-Aldrich. Silicone oil (50 cSt) was purchased from Fisher Scientific. Phosphate-buffered saline (PBS), pH 7.4, was used for in vitro experiments contained 138 mM NaCl, 2.7 mM KCl, and 10 mM sodium phosphate. MRSA (ATCC BA 41), P. aeruginosa (ATCC 9027), and human fibroblasts (CRL 2522) were purchased from American Type Culture Collection. HUVEC were purchased from ThermoFisher Scientific (C0035C). LB broth was obtained from Fisher Bioreagents. LB Agar was purchased from Difco Laboratories. EGM-2 Endothelial Cell Growth Medium-2 BulletKit was purchased from Lonza. Trypsin-EDTA were purchased from Corning (Manassas, VA20109). The Cell Counting Kit-8 (CCK-8) was purchased from Sigma-Aldrich (St. Louis, MO 63103). Eagle's Minimum Essential Medium (EMEM) was purchased from American Type Culture Collection (USA). Fetal bovine serum was procured from VWR (USA). All other cell culture related supplies were purchased from Thermo Fisher Scientific.

### Synthesis of NAP-Thiolactone

The synthesis of NAP-thiolactone was performed according to previously optimized procedures. 48,55 Briefly, 5 g of NAP was dissolved in 10 mL of pyridine in a round-bottom flask and chilled on ice for 1 h. Separately, acetic anhydride (10 mL) was combined with pyridine (10 mL) and also chilled on ice for 1 h. The solutions were then stirred together for 24 h under argon before being rotary evaporated at 60° C. until all of the pyridine and a majority of the acetic anhydride was evaporated. Chloroform (20 mL) was subsequently added to remaining solution and washed with 1 M HCl. The resulting organic layer was dried over anhydrous magnesium sulfate, and the chloroform was evaporated using a desiccator maintained at room temperature. The resulting precipitate was reconstituted in hexanes and kept covered at −20° C. overnight before being filtered, washed with additional hexanes, and left to dry under vacuum.

### Fabrication of NO-PDMS

The fabrication of NO-PDMS was performed according to slightly modified, previously established procedures.^{48, 56} Briefly, hydroxy-terminated PDMS (1.6 g) was dissolved in toluene (8 mL) at room temperature. Separately, (3-aminopropyl)-trimethoxysilane (0.3 g, 1.67 mmol) and dibutyltin dilaurate (2.4 mg) were added to toluene (2 mL). The solutions were then stirred together for 24 h, and then NAP-thiolactone (300 mg, 1.73 mmol) was stirred into the solution for 48 h. Tert-butyl nitrite (500 μL), first chelated by vortexing with a 20 mM cyclam solution three times, was added to nitrosate the NAP-PDMS solution (3 mL), forming a green solution. Additionally, to increase nitrosation and crosslinking efficiency, dodecylbenzene sulfonic acid (0.5 wt % of polymer mass in solution) was added. To fabricate films, the NO-PDMS solution was added to rectangular Teflon molds and dried overnight in the dark at room temperature.

### Liquid-Infusion of NO-PDMS (LI-NO-PDMS) Materials

Liquid-infusion of the fabricated NO-PDMS was achieved based on a previously established procedure.²⁷ Briefly, NO-PDMS samples were immersed and swelled in silicone oil (50 cSt) kept in dark conditions at room temperature. To measure the swelling ratio of the silicone oil-swelled samples (LI-NO-PDMS) over time, the samples were massed before (M₀) and after infusion (M₁), and the following equation was used (Equation 1):

$$Swelling Ratio = \frac{{M}_{1}}{{M}_{0}} \qquad ( Eq . 1 )$$

### Material Characterization

### Sliding Angle Characterization

Prior to measurement, each sample was washed in DI water and dried with a nitrogen stream to ensure the surface was contaminant-free. Sliding angle measurements of 10 μL water droplets were quantified by slowly increasing the angle of LI-NO-PDMS and NO-PDMS samples mounted on a glass slide until water droplets on the surface of the samples slid off. Fifteen measurements on three different coated glass slides at separate positions for each sample type were taken. The sliding angles were determined with a digital protractor. The samples were stored at 37° C. in PBS in an incubator between measurements over 7 days.

### SNAP Leaching Measurements

Cumulative SNAP leaching was measured at 37° C. in 4 mL of PBS. Leachates were collected from both LI-NO-PDMS and NO-PDMS samples over 48 h. Periodically, the optical density of the PBS was measured using a Thermo Scientific Genysis 10 S UV-Vis Spectrophotometer at 340 nm (absorbance maxima of the S-NO bond present in the SNAP molecule) to determine the concentration of SNAP that leached from the samples.⁴⁴ Samples were stored in an incubator maintained at 37° C. between measurements. Blank PBS was used as a control, and a standard curve generated from known concentrations of SNAP-PBS solutions was used to determine the amount of SNAP present in the collected leachates.

### NO Release Measurements

NO release kinetics of LI-NO-PDMS and NO-PDMS samples were measured using a Sievers Chemiluminescence NOA 280i over 30 days. Samples were submerged in PBS in an amber reaction vessel kept at 37° C. using a heated water bath. The PBS was supplemented with 100 mM of EDTA to prevent any unwanted NO catalysis from metallic ions present in the PBS. NO released from the samples was purged from the PBS solution into a chemiluminescent detection chamber using a bubbler and nitrogen sweep gas (200 mL min⁻¹). The purged NO then reacts with ozone present in the chamber, resulting in an excited form of nitrogen dioxide NO^{2*}, which quickly emits a photon used to detect the amount of NO that was originally released from the sample. The average NO flux (x10⁻¹⁰ mol cm⁻² min⁻¹) of each sample type was determined using the NOA constant (mol ppb⁻¹ s⁻¹) and surface area of the sample.

### In Vitro Antimicrobial Efficacy Measurements Over 28 Days

The antimicrobial efficacies of the fabricated materials against Gram-positive and Gram-negative bacterial strains were analyzed after 24 h, 7 days, 14 days, and 28 days of exposure. To determine the initial antimicrobial effects (ex. immediately after insertion of a medical device), a 24 h bacterial adhesion assay was conducted. Isolated strains of MRSA and P. aeruginosa were separately inoculated in 15 mL of LB broth at 37° C. in an incubator shaker at 120 rpm for 15 h. After the inoculation period, the culture was centrifuged at 2500 rpm for 7.5 min, washed with sterile PBS, and centrifuged again at 2500 rpm. After centrifugation, the samples were resuspended in PBS and diluted to achieve a final concentration of ˜10⁸ CFU/mL. The samples (Control PDMS, NO-PDMS, LI-PDMS, and LI-NO-PDMS) were placed in the wells of a 24-well plate (n=4) and incubated with 1 mL of bacterial solution each for 24 h at 37° C. and 120 rpm.

After incubation, the viability of adhered bacterial was assessed by sonicating each sample post-incubation in 1 mL of sterile PBS at 25000 rpm for 60 s followed by vortexing for an additional 30 s. Prior to sonicating, each sample was gently washed with sterile PBS to remove any loosely adhered bacteria. The resulting bacteria-PBS solution was serially diluted, plated on LB agar plates, and kept at 37° C. for 24 h. Colony-forming units (CFUs) were measured to determine the number of viable adhered bacteria normalized to the surface area of each material. The reduction in viability of adhered bacteria was calculated according to the following equation (Equation 2):

$$\% Reduction in adhered bacterial viability = \frac{\frac{{CFU}_{control}}{{cm}^{2}} - \frac{{CFU}_{test}}{{cm}^{2}}}{\frac{{CFU}_{cotrol}}{{cm}^{2}}} \times 100 \qquad ( Eq . 2 )$$

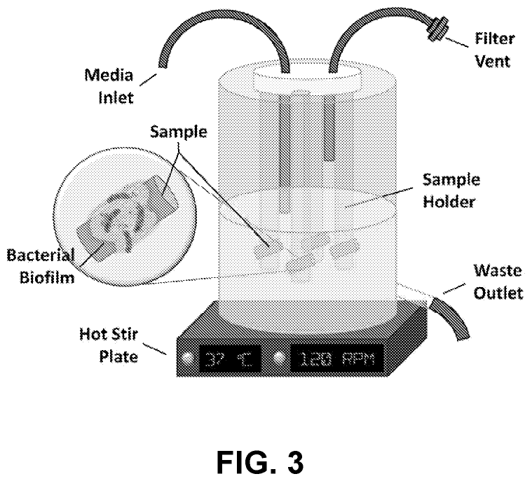

To determine the antimicrobial efficacy of the materials after extended periods of time (7, 14, and 28 days), CDC biofilm bioreactors (FIG. 3) were used to model biofilm formation.⁵⁷ To do so, MRSA and P. aeruginosa were inoculated in LB broth for 15 h broth at 37° C. in an incubator shaker at 120 rpm. The optical density was measured after, and the bacterial culture was diluted to achieve a final concentration of ˜10⁸ CFU/mL and used for incubation with the samples in the bioreactor. The samples were incubated for 1 h and stirred at 200 rpm. After 1 h, the samples were stirred at 120 rpm and supplied with additional nutrient medium (2 g L⁻¹ LB broth) at a rate of ˜1.6 mL min⁻¹. All bioreactors were equipped with an outlet that led to a waste container. The flow was maintained throughout the study until reaching pre-determined endpoints. At termination, the samples were removed and gently washed with sterile PBS. Each sample was sonicated in 1 mL of sterile PBS at 25000 rpm for 60 s followed by vortexing for an additional 30 s. The resulting solution was serially diluted, plated on LB agar plates, and kept at 37° C. for 24 h. Colony-forming units (CFUs) were measured to determine the viability of adhered bacteria normalized to the surface area of each material. The reduction in viability of adhered bacteria was calculated according to the equation above (Equation 2).

### In Vitro Fibrinogen Adsorption Assay

Levels of fibrinogen adsorption were quantified using FITC-labeled human fibrinogen. The labeled fibrinogen was diluted with unlabeled fibrinogen in PBS (pH 7.4) at a 1:10 ratio at a total fibrinogen concentration of 4 mg mL -1 . Samples were incubated in PBS at 37° C. for 30 minutes to allow surface equilibrium. The labeled-unlabeled fibrinogen solution was then added to the solution, resulting in a physiological concentration of 2 mg mL⁻¹. The samples were exposed to fibrinogen for 90 minutes at 37° C. After 90 minutes, the samples were gently washed with PBS to remove any non-bound fibrinogen. Fibrinogen adsorbed onto the surface was then quantified in a plate reader (Biotek, Winooski, Vermont) by measuring the excitation/emission at 495 and 519 nm, and collected data was interpolated using a standard curve. Additionally, a microscope (AMG, Mill Creek, Washington) with a green fluorescence filter was used to image the labeled fibrinogen that adsorbed on the sample surfaces.

### In Vitro Platelet Adhesion Assay

To determine the antiplatelet activity of the samples, an in vitro platelet adhesion assay was executed based on previous studies.^{32,47} Briefly, whole porcine blood (3.8% sodium citrate) was centrifuged at 300 RCF for 13 min to separate and collect platelet-rich plasma (PRP), and again was centrifuged at 4000 RCF for 20 min to separate and collect platelet-poor plasma (PPP). The PRP was diluted with PPP to achieve a final platelet concentration of 2×10⁸ platelets per mL. A CaCl₂ solution was added prior to material exposure at a final concentration of 2 mM. Samples were immersed in 3 mL of the final platelet solution and kept on a rocker for 2 h at 37° C. The samples were then removed and washed with a buffered saline solution. Samples were then kept in 2 v/v % Triton-PBS (500 μL) for 30 min to lyse the adhered platelets. The resulting solutions were dispensed in a 96-well plate, and a Roche Cytotoxicity Detection Kit was prepared to determine the number of adhered platelets. The absorbance was measured at 492 nm using a BioTek Cytation 5 plate reader. Reductions in platelet adhesion were determined using the following formula (Equation 3), where P_{c} is the number of adhered platelets per cm² on control samples and Pₜ is the number of adhered platelets per cm² on test samples:

$$\% Reduction in adhered platelets = \frac{{P}_{c} - {P}_{t}}{{P}_{c}} \times 100 \qquad ( Eq . 3 )$$

### In vitro Whole Blood Exposure

Samples were exposed to porcine whole blood for 60 s and were subsequently gently rinsed with PBS and fixed for scanning electron microscopy (SEM, FEI Teneo). An accelerating voltage of 10.00 kV was used to analyze samples. Prior to SEM imaging, samples were sputter-coated with 10 nm thickness of gold-palladium.

### In vitro Cytotoxicity Measurements

### Culture Method

Both HUVEC and human fibroblasts were revived from cryo stocks stored in liquid nitrogen vapor phase. Fibroblasts were cultured in EMEM supplemented with 10% FBS. HUVEC cells were cultured in EGM-2 supplemented with the following components from the EGM-2 BulletKit: FBS, hydrocortisone, hFGF-B, VEGF, R3-IGF-1, ascorbic acid, hEGF, GA-1000, and heparin. Both cell lines were incubated under 5% CO₂ humid atmosphere at 37° C. Cells were grown to 70-80% confluency then detached via 0.05% trypsin with 5 mM EDTA. Cell counting was done using an EVE TM Cell Counter (NanoEnTek, Waltham, MA USA) with trypan blue cell staining. After cell counting, cells were resuspended and seeded into 96-well, tissue culture-treated polystyrene plates to achieve an initial seeding density of 5,000 cells/well. For each experiment, cells were grown for 24 h before exposure to film leachates.

### Cytotoxicity Evaluation of Film Leachates

Circular coupons of ⅜″ in diameter from each film type were prepared with normalization of surface area across each sample type. Coupons were sterilized via UV exposure for 15 min on each side. Each coupon was then individual submerged in 2 mL of culture media for the respective cell line in a sealed glass vial and incubated for 24 h at 37° C. A separate sample of control media without any film was also incubated at these conditions. After the 24 h incubation, media on seeded plates was aspirated off and replaced with an equal volume of media with leachates from the film samples. For each film coupon, a total of five wells were treated with film leachate. For each experimental run, a total of three coupons per film type were tested. Each plate included experimental controls including unseeded wells with media and seeded wells treated with incubated media without leachate.

Following 24 h incubation under media with film leachates, 10 μL of Cell Counting Kit-8 (CCK-8, Enzo Life Sciences) reagent was added to individual wells. The CCK-8 treated plate was then incubated for an additional 1 h. The CCK-8 assay kit contains WST-8, a tetrazolium salt that is readily reduced by cellular dehydrogenase activity to form a soluble yellow salt detectable at 450 nm. Reference readings at 650 nm were used to correct for noise. Relative viability of leachate-treated cells normalized against untreated cells was then calculated as follows (Equation 4):

$$Relative Cell Viability = \frac{{( AVG {OD}_{450} Read - AVG {OD}_{650} Read )}_{Treatment}}{{( AVG {OD}_{450} Read - AVG {OD}_{650} Read )}_{Control}} \times 100 \qquad ( Eq . 4 )$$

### Statistical Analysis

All data is reported in mean ±SD. A Student's t-test was used to compare the significance between material types during material characterization, and p values <0.05 were considered significant. For in vitro antimicrobial efficacies, cytotoxicity, protein adsorption, and platelet adhesion studies, which all compared the means of four groups, an ANOVA test with post-hoc Tukey was used to determine significance, and p values <0.05 were considered significant.

### Results & Discussion

### Material Optimization & Characterization

### Optimization of Silicone Oil Infusion of NO-PDMS

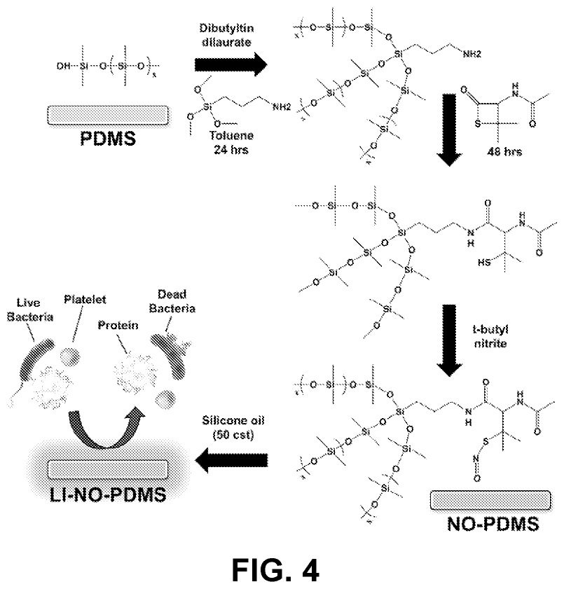

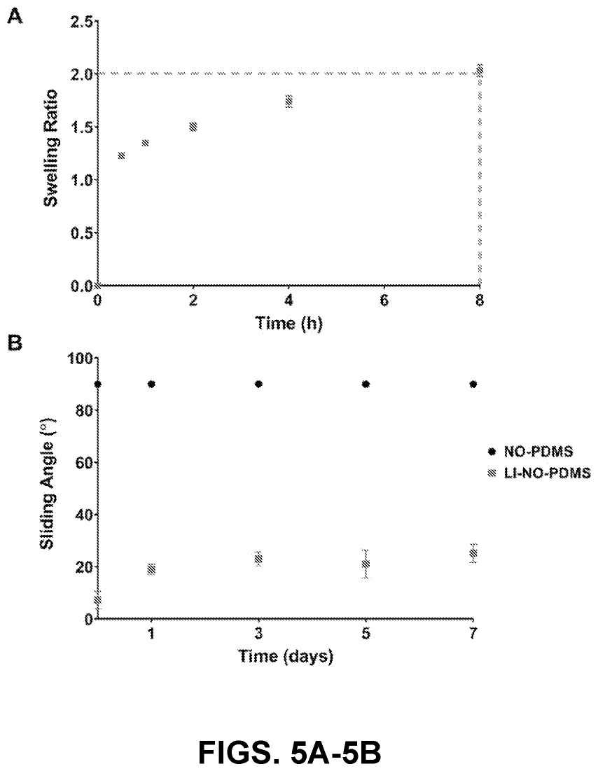

A stable NO-releasing platform was produced to achieve a liquid-infused, antifouling interface via silicone oil infusion (FIG. 4). To optimize the silicone oil infusion process of fabricated NO-PDMS, swelling ratios were measured over time to maximize slippery surface characteristics (FIG. 5A). Liquid infused silicone-based surfaces with a swelling ratio of approximately 2.0 have previously been shown to retain slippery surface characteristics capable of reducing bacterial adhesion and protein adsorption for an extended period of time (>7 days).²⁷ Therefore, in this study, the swelling time of NO-PDMS materials to achieve a swelling ratio of 2.0 was determined. As shown in FIG. 5A, after 8 h of swelling in a silicone oil solution, the materials had a swelling ratio of 2.0±0.1. To ensure that the achieved slippery interface could be maintained under physiological conditions for an extended period, the sliding angles of LI-NO-PDMS and NO-PDMS stored at 37° C. in PBS were compared over 7 days (FIG. 5B). Over the 7-day period, LI-NO-PDMS maintained a sliding angle of 19-25. In comparison, NO-PDMS surfaces exhibited sliding angles >90° throughout the same 7-day period. This demonstrates LI-NO's capability to retain a slippery interface even when stored in an aqueous environment at a physiologically relevant temperature. It can be noted that there was a slight increase in sliding angle between the LI-NO surfaces prior to storage (7.1±)3.3° and after 24 h of storage (19.1±)1.9° (p<0.05). This can be attributed to excess surface oil initially present at the interface that was not completely infused within the polymer. However, after 24 h of incubation, the sliding angle of LI-NO surfaces remained constant, and therefore shows great promise in retaining antifouling functionality desired to enhance hemocompatibility and antimicrobial properties for long-term medical device applications.

### NO Release Measurements & SNAP Leaching Measurements

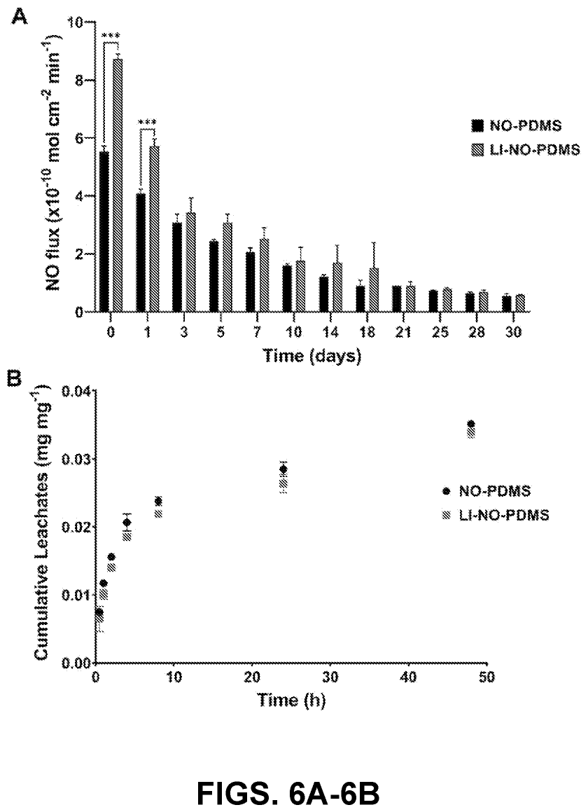

NO is a key endogenous gaseous free radical that is well-documented to mediate cardiovascular hemostasis, immune response, and wound healing.^{37, 60-62} However, the concentration, rate, and longevity of NO release is key in determining the physiological effects NO will have on platelet function, bacterial viability, and surrounding tissue. Healthy endothelium produces NO at an estimated flux of 0.5-4×10⁻¹⁰ mol cm⁻² min⁻¹⁶¹ Therefore, in this work, developed NO-releasing materials were evaluated based on their ability to sustain long-term NO flux>0.5×10⁻¹⁰ mol cm⁻² min⁻¹ to minimize potential thrombus formation and combat infection for long-term, indwelling medical device interfaces. FIG. 6A shows the NO release of both NO-PDMS and LI-NO-PDMS over the course of a 30-day period. The measured NO flux for both the LI-NO-PDMS and NO-PDMS remained>0.5×10⁻¹⁰ mol cm⁻² min⁻¹ flux throughout the 30 days, demonstrating the stability of the material even after extended periods of time under physiological conditions. Previous reports indicate similar NO release kinetics for other SNAP-immobilized polymers.^{48, 63} Interestingly, the LI-NO-PDMS materials had a significantly (p<0.001) higher NO release profile at initially (8.7±0.2×10⁻¹⁰ mol cm⁻² min⁻¹) and after 24 h (5.7±0.2×10⁻¹⁰ mol cm⁻² min⁻¹) compared to NO-PDMS materials both initially (5.5±0.2×10⁻¹⁰ mol cm⁻² min⁻¹) and after 24 h (4.1±0.2×10⁻¹⁰ mol cm⁻² min⁻¹). Because silicone oil acts as a spacer between polymer chains, increased ion interaction with the SNAP groups in the material could result in increased NO release. However, after 3 days, the differences between the samples were minimal. By the end of the study at 30 days, both LI-NO-PDMS and NO-PDMS materials exhibited an NO flux of approximately 0.5×10⁻¹⁰ mol cm⁻² min⁻¹ with no statistical significance between the two sample types (LI-NO-PDMS—0.589±0.002×10⁻¹⁰ mol cm⁻² min⁻¹; NO-PDMS—0.55±0.08×10−10 mol cm⁻² min⁻¹). Therefore, swelling NO-PDMS samples with silicone oil did not affect the long-term NO release characteristics.

The lifetime of materials incorporated with active agents can be severely impacted by leaching when submerged in an aqueous environment. Although some coating technologies have utilized antimicrobial (e.g. antibiotics, chlorine, silver ions) leaching as a means to combat bacterial infection, this method is significantly hindered by the longevity of antimicrobial release, quickly exhausting the available supply and rendering the material vulnerable to infection.⁶⁴ For NO-releasing materials, uncontrolled high doses of NO as a result of NO donor leaching can lead to potential cytotoxicity towards mammalian cells.^{65, 66} Therefore, the SNAP leaching kinetics of LI-NO-PDMS and NO-PDMS materials submerged in PBS (pH 7.4) at 37° C. were measured over 48 h to simulate physiological conditions, which historically has been when most leaching has occurred with NO donor-incorporated materials.^{47, 67} As shown in FIG. 6B, both NO-PDMS and LI-NO-PDMS showed minimal leaching after 48 h and were not significantly different from each other (P>0.05). After 48 h, NO-PDMS and LI-NO-PDMS only leached 0.0351±0.0001 and 0.0339±0.0009 mg of SNAP per mg of substrate, respectively, which is <1% of the total SNAP originally attached. This can be attributed to the fact that the NO donor group is covalently attached to the backbone of the PDMS, virtually eliminating risk of SNAP leaching loss. Therefore, the lack of leachates confirms that the covalent attachment of SNAP results in a stable NO-releasing platform which minimizes potential cytotoxic events that can occur with significant NO donor leaching.

### In vitro Short-Term (24 h) and Long-Term (7-28 days) Antimicrobial Efficacy

With the goal of reducing medical device-associated infections in mind, researchers have developed both active (bactericidal) and passive (antifouling) antimicrobial surface modifications to combat bacterial adhesion and colonization.⁶⁸ However, both passive and active surface strategies have major shortcomings that prevent successful antimicrobial behavior for long-term applications. While able to largely reduce the adhesion of microbes to surfaces, passive surface strategies fail to affect the viability of invading pathogens. Moreover, active surfaces store and release antimicrobial agents that dynamically attack invading bacterial pathogens but have little effect on surface fouling and commonly contain a limited reservoir of antimicrobial agents. However, a platform which contains dual passive-active antimicrobial surface strategies that exhibits both bactericidal and antiadhesive qualities can improve the long-term antimicrobial efficacy of the material. In this work, the NO donor SNAP was covalently attached to medically relevant PDMS to achieve stable NO-releasing properties (active strategy) followed by the liquid infusion of biocompatible silicone oil (passive strategy). The short-term (24 h) and long-term (7-28 days) antimicrobial efficacies were evaluated against Gram-positive MRSA and Gram-negative P. aeruginosa.

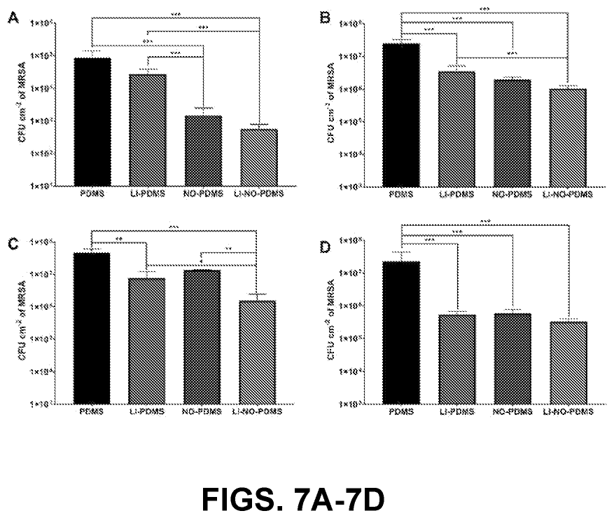

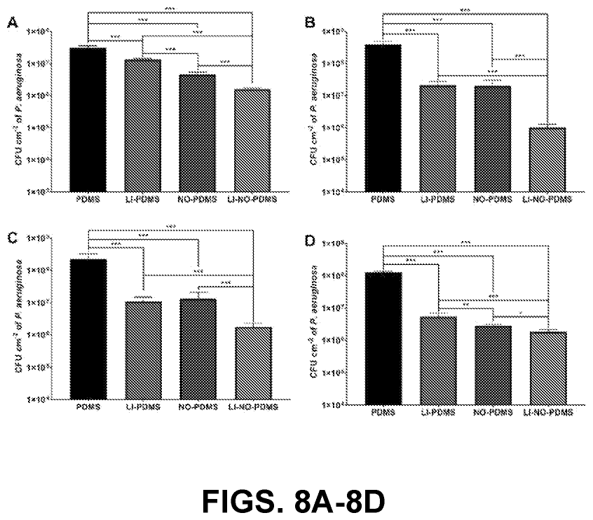

To understand the initial antimicrobial activity of the fabricated materials, samples were exposed to common nosocomial infection-causing pathogens, MRSA and P. aeruginosa, for 24 h under physiological conditions (FIGS. 7A and 8A). Alone, LI-PDMS moderately reduced the number of viable MRSA and P. aeruginosa by 67.8±13.7 and 56.9±3.2%, suggesting that the liquid infusion resulted in decreased bacterial adhesion which has been shown previously by other groups.^{51, 69} Similarly, NO-PDMS also significantly reduced the number of viable adhered MRSA and P. aeruginosa by 98.3±1.2% and 85.1±2.5%. This is expected as NO-releasing materials have historically demonstrated potent antimicrobial and biofilm-dispersing properties with limited resistance.^{70,71} However, LI-NO-PDMS best reduced the viability of both adhered MRSA by 99.3±0.3% and P. aeruginosa by 94.8±0.3% compared to control PDMS after 24 h of exposure. This is in agreement with previous reports assessing the short-term (24 h) antimicrobial efficacy of liquid-infused, NO-releasing platforms, which also found that the combination of liquid-infused (passive) and NO-releasing (active) antimicrobial surface modifications best results in decreased adhered bacterial viability.⁵⁴

To date, two major shortcomings of NO-releasing materials has been high initial burst release coupled with NO donor leaching, quickly depleting the NO reservoir stored in the material and limiting the extent to which the material is able to combat infection for long-term applications. Similarly, superhydrophobic surfaces have been a popular means of achieving antifouling activity for biomedical applications, but due limitations in durability and instability of the trapped air pockets, have failed to improve the hemocompatibility of devices for long-term indwelling medical device applications.^{52, 72} In this work, to extend the antimicrobial surface properties for long-term applications, steady NO-release and robust antifouling technologies achieved through the covalent attachment of SNAP and liquid infusion of silicone oil were deployed. To determine the long-term antimicrobial efficacy, LI-NO-PDMS and control materials were challenged against MRSA and P. aeruginosa in CDC bioreactors for 7, 14, and 28 days. The CDC bioreactor allows for vigorous antimicrobial testing by mimicking shear environments seen within the vasculature, best illustrating the effectiveness of NO-releasing, liquid-infused surfaces in preventing bacterial colonization and biofilm formation. LI-NO-PDMS was significantly effective in reducing the number of viable adhered MRSA and P. aeruginosa at every measured timepoint for up to 28 days compared to untreated materials and exhibited greater reductions in the number of adhered, viable bacteria compared to LI-PDMS and NO-PDMS materials at every timepoint. Table 1 lists the exact % reduction in viable adhered bacteria compared to control PDMS for LI, NO, and LI-NO-PDMS samples. Liquid-infused surfaces have previously resulted in reduction in biofilm formation through decreased bacterial adhesive forces.⁷³ However, liquid-infused surfaces fail to affect bacterial viability. As stated previously, NO-releasing materials have previously demonstrated antimicrobial and biofilm-dispersing capabilities with limited development of resistance through multiple antimicrobial mechanisms including lipid oxidation, enzyme denaturation, and DNA deamination.^{38, 70, 71} Therefore, these results strongly suggest that the stable NO-releasing platform combined with a liquid-infused, slippery interface results in synergistic antimicrobial activity for not only short-term, but also long-term applications.

TABLE 1
Percent reductions in viability of adhered bacteria
when comparing LI-PDMS, NO-PDMS, and LI-NO-PDMS
materials to control PDMS materials.

| | LI-PDMS | NO-PDMS | LI-NO-PDMS |
|---|---|---|---|
| 24 h MRSA (% reduction) | 67.8 ± 13.7 | 98.3 ± 1.2 | 99.3 ± 0.3 |
| 7 d MRSA (%) | 85.8 ± 6.1 | 92.1 ± 1.5 | 95.8 ± 0.9 |
| 14 d MRSA (%) | 78.2 ± 2.0 | 71.3 ± 0.6 | 96.7 ± 1.9 |
| 28 d MRSA (%) | 87.6 ± 3.0 | 86.7 ± 4.1 | 92.6 ± 1.8 |
| 24 h P. aeruginosa (%) | 56.9 ± 3.2 | 85.1 ± 2.5 | 94.8 ± 0.3 |
| 7 d P. aeruginosa (%) | 95.6 ± 2.8 | 94.7 ± 2.4 | 99.7 ± 0.1 |
| 14 d P. aeruginosa (%) | 95.1 ± 1.9 | 94.0 ± 3.5 | 99.2 ± 0.3 |
| 28 d P. aeruginosa (%) | 94.7 ± 1.5 | 97.2 ± 0.4 | 98.2 ± 0.3 |

### Antifouling and Hemocompatible Evaluations

### In vitro Fibrinogen Adsorption

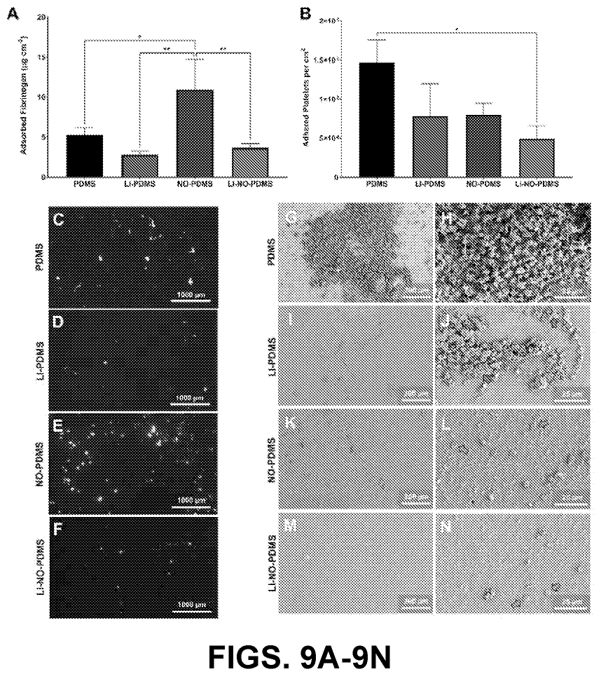

Previous studies have indicated that fibrinogen adsorption onto medical device surfaces propagates undesired platelet adhesion and provides an anchor for bacterial attachment, necessitating antifouling surfaces for medical device applications.¹⁰⁻¹² To determine if the liquid-infused surfaces (LI-PDMS and LI-NO-PDMS) demonstrate improved antifouling properties, samples were incubated with fibrinogen at physiological concentrations (2 mg/mL) for 90 minutes. As shown in FIG. 9A, the liquid infusion of silicone oil resulted in a significant decrease of fibrinogen adsorption (p<0.05, n=3). Adsorption of fibrinogen in LI-PDMS (FIG. 9D) and LI-NO-PDMS (FIG. 9F) samples was reduced by 46.5±8.4% and 30.3±10.2% when compared to control PDMS (FIG. 9C), respectively. Previous reports have similarly shown that liquid infused silicone reduces the attachment of foulants including bacteria and proteins through the creation of a low-adhesion surface.^{27,51} On the other hand, SNAP samples (FIG. 9E) exhibited a significant increase in fibrinogen adsorption by 107.9±71.6% when compared to PDMS. The phenomena of increased protein adsorption on NO-releasing surfaces has been observed in the past.⁷⁴ However, it has been shown that the adsorbed fibrinogen on NO-releasing surfaces is less thrombotic compared to adsorbed fibrinogen on non-NO-releasing surfaces, which suggests that the presence of NO alters the structure of fibrinogen. 49 Further study needs to be conducted to confirm exactly how NO release affects the conformation of fibrinogen.

### In vitro Platelet Adhesion Measurements

Despite material improvements and systemic anticoagulation intervention, device-induced thrombosis remains one of the leading adverse events with indwelling blood-contacting medical devices that often results in consequential side effects including device failure, embolism, extended hospitalization, and increased morbidity and mortality.⁷⁵ Therefore, in order to improve the hemocompatibility of these devices, researchers have begun to develop passive and active surface modifications inspired by nature.²² NO-releasing surfaces, an active strategy that results in a localized production of nitric oxide at the surface, has gained tremendous popularity for improving the antithrombotic characteristics of different materials as a result of NO's role in platelet quiescence.^{29, 30, 76, 77} Moreover, antifouling strategies including liquid-infused super slippery surfaces that minimize biofouling of platelets and plasma proteins have shown promising improvements in hemocompatibility.^{27, 58, 78} Therefore, in this study, the NO donor SNAP was covalently attached to PDMS, a medically-relevant polymer commonly utilized for long-term indwelling medical device applications, followed by liquid infusion of silicone oil, resulting in a sustained NO-releasing platform with an antifouling interface. To assess the antithrombotic characteristics of the resulting material, platelet-rich porcine plasma was exposed for 2 h and the number of adhered platelets was quantified. As shown in FIG. 9B, LI-NO-PDMS significantly reduced the number of platelets adhered to the surface by 66.5±11.2%. Individually, LI-PDMS and NO-PDMS samples reduced the number of adhered platelets by 46.7±27.9% and 45.2±11.2%. Therefore, together the resulting localized NO release and silicone oil infusion resulted in a combined improvement of hemocompatibility. Similar trends of reduced platelet adhesion from NO-releasing surfaces and/or liquid-infused surfaces have been previously characterized.^{27, 50, 59,79-81} In support of these findings, SEM images of materials exposed to porcine whole blood showed that plasma proteins and platelets rapidly adsorbed and adhered to the surface of untreated PDMS surfaces (FIG. 9G, H). However, LI-PDMS (FIG. 9I, J), NO-PDMS (FIG. 9K, L), and LI-NO-PDMS (FIG. 9M, N) samples showed a noticeable reduction in platelets present on the surface. Moreover, while LI-PDMS samples failed to minimize the activation of platelets that did adhere to the surface, both NO-PDMS and LI-NO-PDMS samples showed reduced platelet activation. This is characteristic of NO, which has previously been established to reduce platelet aggregation and activation by inhibiting GPIlb/Illa expression, increasing cGMP activation of protein kinase G, and modulated Ca²⁺ influx.^{32, 82}

### Cytocompatibility of LI-NO Materials

In addition to evaluating efficacy, materials used for medical device applications should be cytocompatible with mammalian cells to ensure safety. In this study, CCK-8 assays were carried out to determine any toxic response towards leachates developed from covalent NO-PDMS and liquid-infused films in human primary cells. To this end, both human BJ fibroblasts and HUVECs were cultured against leachates acquired from films following 24 h incubation in culture media under physiological conditions. These conditions follow the International Organization for Standardization (ISO) 10993-5 for the biological evaluation of medical devices through in vitro tests for cytotoxicity.⁸³

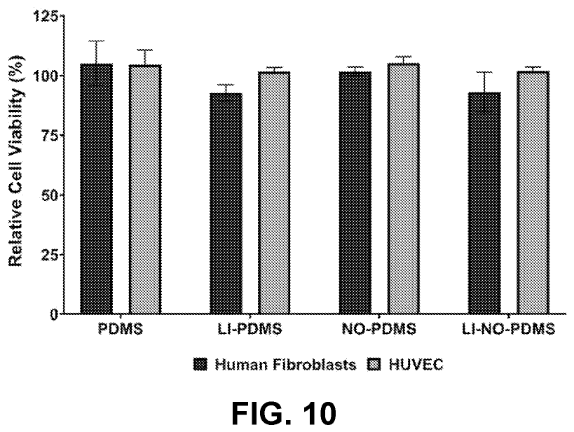

In the case of LI-NO substrates, no appreciable cytotoxic effect was observed over 24 h of leaching and subsequent incubation of BJ fibroblasts and HUVECs (FIG. 10). BJ fibroblasts and HUVECs exhibited an enhanced 105±9.2% and 104±6.1% viability after exposure to PDMS control leachates. These results concur with prior results and may result from trace amounts of primary amine-containing APTMS leachates from the control films. 48 Introduction of immobilized SNAP to these films showed no statistically significant difference in cellular viability (P>0.05). Although liquid infusion led to a minor decline in viability in BJ fibroblast cells (92.6±3.5%), these results were not significant with respect to the PDMS control (P>0.05). Non-volatile medical grade silicone oils have been extensively evaluated in literature with no expected cytotoxicity in indwelling applications provided careful moderation by adjusting infused amounts.⁸⁷ Finally, both NO-PDMS and LI-NO-PDMS exhibited no appreciable effect on cellular viability with respect to PDMS controls in both cell lines (P>0.05). These results are expected, as NAP, the precursor to SNAP, is approved by the FDA for treatment of heavy metal poisoning and cystinuria.^{88, 89} Taken all together, cytocompatibility of covalent-SNAP and liquid-infused substrates motivates interest in their application to indwelling medical devices, especially to combat bacterial infection with the potential for reduced incidence of failure from device occlusion via clot formation.

### Conclusions

In this work, a stable NO-releasing, liquid-infused PDMS (LI-NO-PDMS) platform was fabricated via covalent SNAP immobilization and silicone oil infusion to improve the antimicrobial and antifouling properties for long-term, indwelling medical device applications. The fabrication and optimization of the liquid-infusion protocol resulted in super slippery properties capable of reducing thrombosis and infection. LI-NO-PDMS surfaces maintained a stable, physiologically relevant NO flux (>0.5×10⁻¹⁰ mol cm⁻² mind) for 30 d with <1% of SNAP leaching. As a result, LI-NO-PDMS materials robustly challenged in a series of CDC biofilm reactor studies for 7, 14, and 28 d demonstrated synergistic antimicrobial effects, resulting in up to 99.9% reduction in both adhered MRSA and P. aeruginosa viability and >92% reduction in adhered bacterial viability at any time point and. Herein, we have performed the most exhaustive study to date determining in vitro antimicrobial efficacy of NO-releasing materials using CDC biofilm bioreactors over 28 days. Improved antifouling properties were demonstrated through a substantial reduction in fibrinogen adsorption (30.3±10.2% reduction) and platelet adhesion (66.5±11.2% reduction) to the surface compared to unmodified controls. Finally, leachates extracted from the materials did not exhibit any cytotoxic response towards human fibroblast and endothelial cell lines. In conclusion, these results are substantially promising for the prevention of medical device-induced clotting and biofilm formation. Currently, no study combining a stable NO-releasing platform (>0.5×10⁻¹⁰ mol cm⁻² min d for 30+ days) with slippery surface properties, leaving this technology to be in a unique position to address the widespread problems associated with medical device-related complications.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### REFERENCES

1. Darouiche, R. O., Device-associated infections: a macroproblem that starts with microadherence. Clin Infect Dis 2001, 33 (9), 1567-72.

2. Mermel, L. A.; Allon, M.; Bouza, E.; Craven, D. E.; Flynn, P.; O′Grady, N. P.; Raad, II; Rijnders, B. J.; Sherertz, R. J.; Warren, D. K., Clinical practice guidelines for the diagnosis and management of intravascular catheter-related infection: 2009 Update by the Infectious Diseases Society of America. Clin Infect Dis 2009, 49 (1), 1-45.

3. Stewart, P. S., Mechanisms of antibiotic resistance in bacterial biofilms. Int J Med Microbiol 2002, 292 (2), 107-13.

4. Burns, K. E. A.; McLaren, A., Catheter-related right atrial thrombus and pulmonary embolism: A case report and systematic review of the literature. Can Respir J 2009, 16 (5), 163-165.

5. White, D.; Woller, S. C.; Stevens, S. M.; Collingridge, D. S.; Chopra, V.; Fontaine, G. V., Comparative thrombosis risk of vascular access devices among critically ill medical patients. Thrombosis Research 2018, 172, 54-60.

6. Dillon, P. A.; Foglia, R. P., Complications associated with an implantable vascular access device. Journal of pediatric surgery 2006, 41 (9), 1582-1587.

7. Roberts, T. R.; Harea, G. T.; Singha, P.; Sieck, K. N.; Beely, B. M.; Wendorff, D. S.; Choi, J. H.; Ande, S.; Handa, H.; Batchinsky, A. I., Heparin-Free Extracorporeal Life Support Using Tethered Liquid Perfluorocarbon: A Feasibility and Efficacy Study. ASAIO J 2020, 66 (7), 809-817.

8. Warkentin, T. E., Heparin-induced thrombocytopenia. Critical Decisions in Thrombosis and Haemostasis. BC Decker Inc. Hamilton,. Pagg 1998, 100-8.

9. Fidlar, E.; Jaques, L., The effect of commercial heparin on the platelet count. The Journal of laboratory and clinical medicine 1948, 33 (11), 1410-1423.

10. Charville, G. W.; Hetrick, E. M.; Geer, C. B.; Schoenfisch, M. H., Reduced bacterial adhesion to fibrinogen-coated substrates via nitric oxide release. Biomaterials 2008, 29 (30), 4039-44.

11. Horbett, T. A., Fibrinogen adsorption to biomaterials. J Biomed Mater Res A 2018, 106 (10), 2777-2788.

12. Mohammad, S. F.; Topham, N. S.; Burns, G. L.; Olsen, D. B., Enhanced bacterial adhesion on surfaces pretreated with fibrinogen and fibronectin. ASAIO Trans 1988, 34 (3), 573-7.

13. Liu, L.; Shi, H.; Yu, H.; Yan, S.; Luan, S., The recent advances in surface antibacterial strategies for biomedical catheters. Biomater Sci 2020, 8 (15), 4095-4108.

14. Singha, P.; Locklin, J.; Handa, H., A review of the recent advances in antimicrobial coatings for urinary catheters. Acta Biomater 2017, 50, 20-40.

15. Johnson, J. R.; Johnston, B.; Kuskowski, M. A., In vitro comparison of nitrofurazone-and silver alloy-coated foley catheters for contact-dependent and diffusible inhibition of urinary tract infection-associated microorganisms. Antimicrob Agents Chemother 2012, 56 (9), 4969-72.

16. Johnson, J. R.; Kuskowski, M. A.; Wilt, T. J., Systematic review: antimicrobial urinary catheters to prevent catheter-associated urinary tract infection in hospitalized patients. Ann Intern Med 2006, 144 (2), 116-26.

17. Kowalczuk, D.; Ginalska, G.; Piersiak, T.; Miazga-Karska, M., Prevention of biofilm formation on urinary catheters: comparison of the sparfloxacin-treated long-term antimicrobial catheters with silver-coated ones. J Biomed Mater Res B Appl Biomater 2012, 100 (7), 1874-82.

18. Morris, N. S.; Stickler, D. J., Encrustation of indwelling urethral catheters by Proteus mirabilis biofilms growing in human urine. J Hosp Infect 1998, 39 (3), 227-34.

19. Riley, D. K.; Classen, D. C.; Stevens, L. E.; Burke, J. P., A large randomized clinical trial of a silver-impregnated urinary catheter: lack of efficacy and staphylococcal superinfection. Am J Med 1995, 98 (4), 349-56.

20. Antonelli, M.; De Pascale, G.; Ranieri, V.; Pelaia, P.; Tufano, R.; Piazza, O.; Zangrillo, A.; Ferrario, A.; De Gaetano, A.; Guaglianone, E., Comparison of triple-lumen central venous catheters impregnated with silver nanoparticles (AgTive®) vs conventional catheters in intensive care unit patients. Journal of Hospital Infection 2012, 82 (2), 101-107.

21. Stevens, K. N.; Crespo-Biel, O.; van den Bosch, E. E.; Dias, A. A.; Knetsch, M. L.; Aldenhoff, Y. B.; van der Veen, F. H.; Maessen, J. G.; Stobberingh, E. E.; Koole, L. H., The relationship between the antimicrobial effect of catheter coatings containing silver nanoparticles and the coagulation of contacting blood. Biomaterials 2009, 30 (22), 3682-90.

22. Maitz, M. F.; Martins, M. C. L.; Grabow, N.; Matschegewski, C.; Huang, N.; Chaikof, E. L.; Barbosa, M. A.; Werner, C.; Sperling, C., The blood compatibility challenge. Part 4: Surface modification for hemocompatible materials: Passive and active approaches to guide blood-material interactions. Acta Biomater 2019, 94, 33-43.

23. Lavery, K. S.; Rhodes, C.; McGraw, A.; Eppihimer, M. J., Anti-thrombotic technologies for medical devices. Adv Drug Deliv Rev 2017, 112, 2-11.

24. Harding, J. L.; Metz, J. M.; Reynolds, M. M., A Tunable, Stable, and Bioactive MOF Catalyst for Generating a Localized Therapeutic from Endogenous Sources. Advanced Functional Materials 2014, 24 (47), 7503-7509.

25. Ashcraft, M.; Douglass, M.; Chen, Y.; Handa, H., Combination strategies for antithrombotic biomaterials: an emerging trend towards hemocompatibility. Biomater Sci 2021, 9 (7), 2413-2423.

26. Ergene, C.; Yasuhara, K.; Palermo, E. F., Biomimetic antimicrobial polymers: recent advances in molecular design. Polym Chem-Uk 2018, 9 (18), 2407-2427.

27. Goudie, M. J.; Pant, J.; Handa, H., Liquid-infused nitric oxide-releasing (LINORel) silicone for decreased fouling, thrombosis, and infection of medical devices. Sci Rep 2017, 7 (1), 13623.

28. May, R. M.; Magin, C. M.; Mann, E. E.; Drinker, M. C.; Fraser, J. C.; Siedlecki, C. A.; Brennan, A. B.; Reddy, S. T., An engineered micropattern to reduce bacterial colonization, platelet adhesion and fibrin sheath formation for improved biocompatibility of central venous catheters. Clin Transl Med 2015, 4.

29. Brisbois, E. J.; Kim, M.; Wang, X.; Mohammed, A.; Major, T. C.; Wu, J.; Brownstein, J.; Xi, C.; Handa, H.; Bartlett, R. H.; Meyerhoff, M. E., Improved Hemocompatibility of Multilumen Catheters via Nitric Oxide (NO) Release from S-Nitroso-N-acetylpenicillamine (SNAP) Composite Filled Lumen. ACS Appl Mater Interfaces 2016, 8 (43), 29270-29279.

30. Brisbois, E. J.; Major, T. C.; Goudie, M. J.; Bartlett, R. H.; Meyerhoff, M. E.; Handa, H., Improved hemocompatibility of silicone rubber extracorporeal tubing via solvent swelling-impregnation of S-nitroso-N-acetylpenicillamine (SNAP) and evaluation in rabbit thrombogenicity model. Acta Biomater 2016, 37, 111-9.

31. Devine, R.; Goudie, M. J.; Singha, P.; Schmiedt, C.; Douglass, M.; Brisbois, E. J.; Handa, H., Mimicking the Endothelium: Dual Action Heparinized Nitric Oxide Releasing Surface. ACS Appl Mater Interfaces 2020, 12 (18), 20158-20171.

32. Douglass, M.; Hopkins, S.; Pandey, R.; Singha, P.; Norman, M.; Handa, H., S-Nitrosoglutathione-Based Nitric Oxide-Releasing Nanofibers Exhibit Dual Antimicrobial and Antithrombotic Activity for Biomedical Applications. Macromol Biosci 2020, e2000248.

33. Estes, L. M.; Singha, P.; Singh, S.; Sakthivel, T. S.; Garren, M.; Devine, R.; Brisbois, E. J.; Seal, S.; Handa, H., Characterization of a nitric oxide (NO) donor molecule and cerium oxide nanoparticle (CNP) interactions and their synergistic antimicrobial potential for biomedical applications. J Colloid Interface Sci 2020.

34. Hibbard, H. A. J.; Reynolds, M. M., Synthesis of novel nitroreductase enzyme-activated nitric oxide prodrugs to site-specifically kill bacteria. Bioorg Chem 2019, 93, 103318.

35. Maloney, S. E.; McGrath, K. V.; Ahonen, M. J. R.; Soliman, D. S.; Feura, E. S.; Hall, H. R.; Wallet, S. M.; Maile, R.; Schoenfisch, M. H., Nitric Oxide-Releasing Hyaluronic Acid as an Antibacterial Agent for Wound Therapy. Biomacromolecules 2020.

36. Tuteja, N.; Chandra, M.; Tuteja, R.; Misra, M. K., Nitric Oxide as a Unique Bioactive Signaling Messenger in Physiology and Pathophysiology. J Biomed Biotechnol 2004, 2004 (4), 227-237.

37. Garren, M. R.; Ashcraft, M.; Qian, Y.; Douglass, M.; Brisbois, E. J.; Handa, H., Nitric oxide and viral infection: Recent developments in antiviral therapies and platforms. Appl Mater Today 2021, 22.

38. Schairer, D. O.; Chouake, J. S.; Nosanchuk, J. D.; Friedman, A. J., The potential of nitric oxide releasing therapies as antimicrobial agents. Virulence 2012, 3 (3), 271-9.

39. Wang, G. R.; Zhu, Y.; Halushka, P. V.; Lincoln, T. M.; Mendelsohn, M. E., Mechanism of platelet inhibition by nitric oxide: in vivo phosphorylation of thromboxane receptor by cyclic GMP-dependent protein kinase. Proc Natl Acad Sci U S A 1998, 95 (9), 4888-4893.

40. Ghalei, S.; Mondal, A.; Hopkins, S.; Singha, P.; Devine, R.; Handa, H., Silk Nanoparticles: A Natural Polymeric Platform for Nitric Oxide Delivery in Biomedical Applications. ACS Appl Mater Interfaces 2020.

41. Feura, E. S.; Yang, L.; Schoenfisch, M. H., Antibacterial activity of nitric oxide-releasing carboxymethylcellulose against periodontal pathogens. J Biomed Mater Res A 2020.

42. Suchyta, D. J.; Schoenfisch, M. H., Encapsulation of N-Diazeniumdiolates within Liposomes for Enhanced Nitric Oxide Donor Stability and Delivery. Mol Pharm 2015, 12 (10), 3569-74.

43. Mondal, A.; Devine, R.; Estes, L.; Manuel, J.; Singha, P.; Mancha, J.; Palmer, M.; Handa, H., Highly hydrophobic polytetrafluoroethylene particle immobilization via polydopamine anchor layer on nitric oxide releasing polymer for biomedical applications. J Colloid Interface Sci 2020.

44. Douglass, M. E.; Goudie, M. J.; Pant, J.; Singha, P.; Hopkins, S.; Devine, R.; Schmiedt, C. W.; Handa, H., Catalyzed nitric oxide release via Cu nanoparticles leads to an increase in antimicrobial effects and hemocompatibility for short-term extracorporeal circulation. ACS Appl. Bio Mater. 2019, 2 (6), 2539-2548.

45. Frost, M. C.; Meyerhoff, M. E., Synthesis, characterization, and controlled nitric oxide release from S-nitrosothiol-derivatized fumed silica polymer filler particles. J Biomed Mater Res A 2005, 72 (4), 409-19.

46. Ratanatawanate, C.; Chyao, A.; Balkus, K. J., Jr., 5-nitrosocysteine-decorated PbS QDs/TiO2 nanotubes for enhanced production of singlet oxygen. J Am Chem Soc 2011, 133 (10), 3492-7.

47. Mondal, A.; Douglass, M.; Hopkins, S. P.; Singha, P.; Tran, M.; Handa, H.; Brisbois, E. J., Multifunctional S-Nitroso-N-acetylpenicillamine-lncorporated Medical-Grade Polymer with Selenium Interface for Biomedical Applications. ACS Appl Mater Interfaces 2019, 11 (38), 34652-34662.

48. Hopkins, S. P.; Pant, J.; Goudie, M. J.; Schmiedt, C.; Handa, H., Achieving Long-Term Biocompatible Silicone via Covalently Immobilized S-Nitroso- N-acetylpenicillamine (SNAP) That Exhibits 4 Months of Sustained Nitric Oxide Release. ACS Appl Mater Interfaces 2018, 10 (32), 27316-27325.

49. Lantvit, S. M.; Barrett, B. J.; Reynolds, M. M., Nitric oxide releasing material adsorbs more fibrinogen. Journal of Biomedical Materials Research Part A 2013, 101 (11), 3201-3210.

50. Leslie, D. C.; Waterhouse, A.; Berthet, J. B.; Valentin, T. M.; Watters, A. L.; Jain, A.; Kim, P.; Hatton, B. D.; Nedder, A.; Donovan, K.; Super, E. H.; Howell, C.; Johnson, C. P.; Vu, T. L.; Bolgen, D. E.; Rifai, S.; Hansen, A. R.; Aizenberg, M.; Super, M.; Aizenberg, J.; lngber, D. E., A bioinspired omniphobic surface coating on medical devices prevents thrombosis and biofouling. Nat Biotechnol 2014, 32 (11), 1134-40.

51. MacCallum, N.; Howell, C.; Kim, P.; Sun, D.; Friedlander, R.; Ranisau, J.; Ahanotu, O.; Lin, J. J.; Vena, A.; Hatton, B.; Wong, T. S.; Aizenberg, J., Liquid-Infused Silicone As a Biofouling-Free Medical Material. Acs Biomater Sci Eng 2015, 1 (1), 43-51.

52. Wang, C. H.; Guo, Z. G., A comparison between superhydrophobic surfaces (SHS) and slippery liquid-infused porous surfaces (SLIPS) in application. Nanoscale 2020, 12 (44), 22398-22424.

53. Chug, M. K.; Feit, C.; Brisbois, E. J., Increasing the lifetime of insulin cannula with antifouling and nitric oxide releasing properties. ACS Appl. Bio Mater. 2019, 2 (12), 5965-5975.

54. Homeyer, K. H.; Goudie, M. J.; Singha, P.; Handa, H., Liquid-Infused Nitric-Oxide-Releasing Silicone Foley Urinary Catheters for Prevention of Catheter-Associated Urinary Tract Infections. ACS Biomaterials Science & Engineering 2019, 5 (4), 2021-2029.

55. Moynihan, H. A.; Roberts, S. M., Preparation of Some Novel S-Nitroso Compounds as Potential Slow-Release - Agents of Nitric-Oxide in-Vivo. J Chem Soc Perk T 1 1994, (7), 797-805.

56. Gierke, G. E.; Nielsen, M.; Frost, M. C., S-Nitroso-N-acetyl-D-penicillamine covalently linked to polydimethylsiloxane (SNAP-PDMS) for use as a controlled photoinitiated nitric oxide release polymer. Sci Technol Adv Mater 2011, 12 (5), 055007.

57. Goeres, D. M.; Loetterle, L. R.; Hamilton, M. A.; Murga, R.; Kirby, D. W.; Donlan, R. M., Statistical assessment of a laboratory method for growing biofilms. Microbiology 2005, 151 (3), 757-762.

58. Sotiri, I.; Overton, J. C.; Waterhouse, A.; Howell, C., Immobilized liquid layers: A new approach to anti-adhesion surfaces for medical applications. Exp Biol Med (Maywood) 2016, 241 (9), 909-18.

59. Yuan, S.; Li, Z.; Song, L.; Shi, H.; Luan, S.; Yin, J., Liquid-Infused Poly(styrene-b-isobutylene-b-styrene) Microfiber Coating Prevents Bacterial Attachment and Thrombosis. ACS Appl Mater Interfaces 2016, 8 (33), 21214-20.

60. Tripathi, M. K.; Kartawy, M.; Amal, H., The role of nitric oxide in brain disorders: Autism spectrum disorder and other psychiatric, neurological, and neurodegenerative disorders. Redox Biol 2020, 34, 101567.

61. Vaughn, M. W.; Kuo, L.; Liao, J. C., Estimation of nitric oxide production and reaction rates in tissue by use of a mathematical model. Am J Physiol 1998, 274 (6), H2163-76.

62. Yang, Y.; Qi, P. K.; Yang, Z. L.; Huang, N., Nitric oxide based strategies for applications of biomedical devices. Biosurface and Biotribology 2015, 1 (3), 177-201.

63. Hopkins, S. P.; Frost, M. C., Synthesis and Characterization of Controlled Nitric Oxide Release from S-Nitroso-N-Acetyl-d-Penicillamine Covalently Linked to Polyvinyl Chloride (SNAP-PVC). Bioengineering (Basel) 2018, 5(3).

64. Yatvin, J.; Gao, J.; Locklin, J., Durable defense: robust and varied attachment of non-leaching poly“-onium” bactericidal coatings to reactive and inert surfaces. Chem Commun (Camb) 2014, 50 (67), 9433-42.

65. Kolb, H.; Kolb-Bachofen, V., Nitric oxide in autoimmune disease: cytotoxic or regulatory mediator? Immunol Today 1998, 19 (12), 556-61.

66. Singha, P.; Workman, C. D.; Pant, J.; Hopkins, S. P.; Handa, H., Zinc-oxide nanoparticles act catalytically and synergistically with nitric oxide donors to enhance antimicrobial efficacy. J Biomed Mater Res A 2019, 107 (7), 1425-1433.

67. Brisbois, E. J.; Handa, H.; Major, T. C.; Bartlett, R. H.; Meyerhoff, M. E., Long-term nitric oxide release and elevated temperature stability with S-nitroso-N-acetylpenicillamine (SNAP)-doped Elast-eon E2As polymer. Biomaterials 2013, 34 (28), 6957-66.

68. Rigo, S.; Cai, C.; Gunkel-Grabole, G.; Maurizi, L.; Zhang, X.; Xu, J.; Palivan, C. G., Nanoscience-Based Strategies to Engineer Antimicrobial Surfaces. Adv Sci (Weinh) 2018, 5 (5), 1700892.

69. Wei, C.; Zhang, G.; Zhang, Q.; Zhan, X.; Chen, F., Silicone Oil-Infused Slippery Surfaces Based on Sol-Gel Process-Induced Nanocomposite Coatings: A Facile Approach to Highly Stable Bioinspired Surface for Biofouling Resistance. ACS Appl Mater Interfaces 2016, 8 (50), 34810-34819.

70. Barraud, N.; Hassett, D. J.; Hwang, S. H.; Rice, S. A.; Kjelleberg, S.; Webb, J. S., Involvement of nitric oxide in biofilm dispersal of Pseudomonas aeruginosa. J Bacteriol 2006, 188 (21), 7344-53.

71. Singha, P.; Goudie, M. J.; Liu, Q.; Hopkins, S.; Brown, N.; Schmiedt, C. W.; Locklin, J.; Handa, H., Multipronged Approach to Combat Catheter-Associated Infections and Thrombosis by Combining Nitric Oxide and a Polyzwitterion: a 7 Day In Vivo Study in a Rabbit Model. ACS Appl Mater Interfaces 2020, 12 (8), 9070-9079.

72. Toes, G. J.; van Muiswinkel, K. W.; van Oeveren, W.; Suurmeijer, A. J.; Timens, W.; Stokroos, I.; van den Dungen, J. J., Superhydrophobic modification fails to improve the performance of small diameter expanded polytetrafluoroethylene vascular grafts. Biomaterials 2002, 23 (1), 255-62.

73. Doll, K.; Yang, I.; Fadeeva, E.; Kommerein, N.; Szafranski, S. P.; Bei der VVieden, G.; Greuling, A.; Winkel, A.; Chichkov, B. N.; Stumpp, N. S.; Stiesch, M., Liquid-Infused Structured Titanium Surfaces: Antiadhesive Mechanism to Repel Streptococcus oralis Biofilms. ACS Appl Mater Interfaces 2019, 11 (26), 23026-23038.

74. Devine, R.; Singha, P.; Handa, H., Versatile biomimetic medical device surface: hydrophobin coated, nitric oxide-releasing polymer for antimicrobial and hemocompatible applications. Biomaterials science 2019.

75. Jaffer, I. H.; Weitz, J. I., The blood compatibility challenge. Part 1: Blood-contacting medical devices: The scope of the problem. Acta Biomater 2019, 94, 2-10.

76. Major, T. C.; Handa, H.; Brisbois, E. J.; Reynolds, M. M.; Annich, G. M.; Meyerhoff, M. E.; Bartlett, R. H., The mediation of platelet quiescence by NO-releasing polymers via cGMP-induced serine 239 phosphorylation of vasodilator-stimulated phosphoprotein. Biomaterials 2013, 34 (33), 8086-96.

77. Zhang, H.; Annich, G. M.; Miskulin, J.; Osterholzer, K.; Merz, S. I.; Bartlett, R. H.; Meyerhoff, M. E., Nitric oxide releasing silicone rubbers with improved blood compatibility: preparation, characterization, and in vivo evaluation. Biomaterials 2002, 23 (6), 1485-94.

78. Epstein, A. K.; Wong, T. S.; Belisle, R. A.; Boggs, E. M.; Aizenberg, J., Liquid-infused structured surfaces with exceptional anti-biofouling performance. Proc Natl Acad Sci U S A 2012, 109 (33), 13182-7.

79. Goudie, M. J.; Singha, P.; Hopkins, S. P.; Brisbois, E. J.; Handa, H., Active Release of an Antimicrobial and Antiplatelet Agent from a Nonfouling Surface Modification. ACS Appl Mater Interfaces 2019, 11 (4), 4523-4530.

80. Wong, T. S.; Kang, S. H.; Tang, S. K.; Smythe, E. J.; Hatton, B. D.; Grinthal, A.; Aizenberg, J., Bioinspired self-repairing slippery surfaces with pressure-stable omniphobicity. Nature 2011, 477 (7365), 443-7.

81. Yuan, S.; Luan, S.; Yan, S.; Shi, H.; Yin, J., Facile Fabrication of Lubricant-Infused Wrinkling Surface for Preventing Thrombus Formation and Infection. ACS Appl Mater Interfaces 2015, 7 (34), 19466-73.

82. Simon-Walker, R.; Romero, R.; Stayer, J. M.; Zang, Y.; Reynolds, M. M.; Popat, K. C.; Kipper, M. J., Glycocalyx-Inspired Nitric Oxide-Releasing Surfaces Reduce Platelet Adhesion and Activation on Titanium. Acs Biomater Sci Eng 2017, 3 (1), 68-77.

83. Wallin, R. F.; Arscott, E. F., A practical guide to ISO 10993-5: Cytotoxicity. Medical Device and Diagnostic Industry 1998, 20, 96-98.

84. Han, G.; Nguyen, L. N.; Macherla, C.; Chi, Y.; Friedman, J. M.; Nosanchuk, J. D.; Martinez, L. R., Nitric Oxide—Releasing Nanoparticles Accelerate Wound Healing by Promoting Fibroblast Migration and Collagen Deposition. The American Journal of Pathology 2012, 180 (4), 1465-1473.

85. Ghalei, S.; Li, J.; Douglass, M.; Garren, M.; Handa, H., Synergistic Approach to Develop Antibacterial Electrospun Scaffolds Using Honey and S-Nitroso-N-acetyl Penicillamine. Acs Biomater Sci Eng 2021.

86. Roberts, T. R.; Garren, M.; Handa, H.; Batchinsky, A. I., Toward an Artificial Endothelium: Development of Blood-Compatible Surfaces for Extracorporeal Life Support. J Trauma Acute Care Surg 2020.

87. Howell, C.; Grinthal, A.; Sunny, S.; Aizenberg, M.; Aizenberg, J., Designing Liquid-Infused Surfaces for Medical Applications: A Review. Adv Mater 2018, 30 (50), e1802724.

88. Kark, R. A.; Poskanzer, D. C.; Bullock, J. D.; Boylen, G., Mercury poisoning and its treatment with N-acetyl-D,L-penicillamine. N Engl J Med 1971, 285 (1), 10-6.

89. Stephens, A. D.; Watts, R. W., The treatment of cystinuria with N-acetyl-D-penicillamine, a comparison with the results of D-penicillamine treatment. Q J Med 1971, 40 (159), 355-70.

We claim:

1. A composition comprising
(a) a nitric oxide releasing material comprising a (i) a polysiloxane network and (ii) a plurality of nitric oxide-donating moieties covalently bonded to the polysiloxane network; and
(b) a silicone oil.

2. The composition of claim 1, wherein the nitric oxide-donating moiety comprises an S-nitrosothiol

3. The composition of claim 1, wherein the nitric oxide-donating moiety is a residue of S-nitroso-N-acetyl-penicillamine, S-nitroso-N-acetyl cysteine, S-nitroso-N-acetyl cysteamine, S-nitrosoglutathione, methyl S-nitrosothioglycolate, and a derivative thereof.

4. The composition of claim 1, wherein the polysiloxane network comprises a polysiloxane crosslinked with an amine-functionalized crosslinker.

5. The composition of claim 4, wherein the polysiloxane comprises a polydimethylsiloxane, a polydiethylsiloxane, a polydipropylsiloxane, or a polydiphenylsiloxane.

6. The composition of claim 5, wherein the polysiloxane has a kinematic viscosity of about 2,500 cSt to about 4,000 cSt.

7. The composition of claim 1, wherein the nitric oxide-releasing material is produced the method comprising:
crosslinking a polysiloxane with an amine-functionalized crosslinker to produce a polysiloxane network
covalently attaching a thiolactone to the polysiloxane network to produce a thiol-functionalized polysiloxane network; and
nitrosating a thiol group in the thiol-functionalized polysiloxane network in the presence of an organic acid to produce the nitric oxide-releasing material.

8. The composition of claim 7, wherein the organic acid comprises an organic sulfonic acid.

9. The composition of claim 7, wherein the organic acid comprises dodecylbenzene sulfonic acid, dinonylnaphthalenedisulfonic acid, 4-octylbenzenesulfonic acid, acetic acid, formic acid, or lactic acid.

10. The composition of claim 7, wherein the organic acid is dodecylbenzene sulfonic acid.

11. The composition of claim 7, wherein the organic acid is in the amount of from about 0.1 weight percent to about 2 weight percent of the polysiloxane network.

12. The composition of claim 7, wherein the polysiloxane comprises a hydroxy-terminated polysiloxane.

13. The composition of claim 7, wherein the amine-functionalized crosslinker has the structure 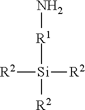
where R¹ is selected from a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy;
where each occurrence of R² is hydroxy or alkoxy.

14. The composition of claim 13, wherein each occurrence of R² is a hydroxy, methoxy or ethoxy.

15. The composition of claim 13, wherein R¹ is a substituted or unsubstituted C1-C₁₂ alkyl or a substituted or unsubstituted C₁-C₁₂ aminoalkyl.

16. The composition of claim 13, wherein R¹ is methylene, ethylene, propylene, or butylene.

17. The composition of claim 7, wherein the thiolactone has the structure 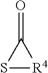
where R⁴ is a substituted or unsubstituted C₁-C₁₂ alkyl.

18. The composition of claim 7, wherein the thiolactone has the structure 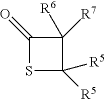
where each occurrence of R⁵ is independently hydrogen, a hydroxyl group, a substituted or unsubstituted C₁-C₆ alkyl group, substituted or unsubstituted C1-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, or a substituted or unsubstituted C₁-C₆ heteroalkoxy group;
R⁶ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, or a substituted or unsubstituted C₁-C₆ heteroalkoxy group; and
R⁷ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, a substituted or unsubstituted C₁-C₆ heteroalkoxy group, or an amide group of the formula —NHC(O)R⁸, wherein R⁸ is a substituted or unsubstituted C₁-C₆ alkyl group, substituted or unsubstituted C₁-C₆ heteroalkyl group.

19. The composition of claim 7, wherein the thiolactone is selected from the group consisting of N-acetylcysteine thiolactone, N-acetyl-homocysteine thiolactone, homocysteine thiolactone, and butyryl-homocysteine thiolactone.

20. The composition of claim 1, wherein the nitric oxide-donating moiety is a residue of S-nitroso-N-acetyl-penicillamine, S-nitroso-N-acetyl cysteine, S-nitroso-N-acetyl cysteamine, S-nitrosoglutathione, or methyl S-nitrosothioglycolate.

21. The composition of claim 1, wherein the nitric oxide-donating moieties are present in an amount from about 0.15 micromoles per milligram of the polymer matrix to about 0.80 micromoles per milligram of the polysiloxane network.

22. The composition of claim 1, wherein the silicone oil has a viscosity of from about 10 cSt to about 500 cSt.

23. The composition of claim 1, wherein nitric oxide releasing material and silicone oil are in an amount sufficient such that the composition has a swelling ratio of from about 0.5 to about 3.

24. The composition of claim 1, wherein nitric oxide is released from the composition for at least 30 days at 37° C.

25. The composition of claim 1, wherein the composition comprises an admixture of the nitric oxide releasing material and silicone oil.

26. The composition of claim 1, wherein the nitric oxide releasing material is impregnated with the silicone oil.

27. An article comprising at least one surface, wherein the at least one surface is coated with the composition in any one of claims 1-26.

28. An article comprising one or more components fabricated with the composition in any one of claims 1-26.

29. The article of claim 27, wherein the article comprises a medical device.

30. The article of claim 29, wherein the device is an implantable device.

31. The article of claim 29, wherein the device is selected from the group consisting of: a vascular catheter, a urinary catheter, other catheters, a coronary stent, a wound dressing, and a vascular graft.

32. A method of making an article, the method comprising
(a) applying a nitric oxide releasing material to at least one surface of the article to produce a first coated article; and
(b) applying silicone oil to the first coated article.

33. The method of claim 32, wherein the nitric oxide releasing material has a thickness of from about 0.1 mm to about 5 mm.

34. The method of claim 32, wherein the first coated article is dipped into the silicone oil from about 1 hour to about 12 hours.

35. A method of preventing bacterial growth on a surface of an article, the method comprising applying the composition in any one of claims 1-26 to the surface.

36. A method of preventing biofilm formation on a surface of an article, the method comprising applying the composition in any one of claims 1-26 to the surface.

37. A method of preventing fibrinogen formation on a surface of an article, the method comprising applying the composition in any one of claims 1-26 to the surface.

38. A method of preventing fibrinogen formation on a surface of an article, the method comprising applying the composition in any one of claims 1-26 to the surface.
